(19) United States
(12) Patent Application Publication
(10) Pub. No.: US 2025/0250331 A1
(43) Pub. Date: Aug. 7, 2025

(54) ANTI-AGE ANTIBODIES FOR TREATING NEURODEGENERATIVE DISORDERS

(71) Applicant: Siwa Corporation, Chicago, IL (US)

(72) Inventor: Lewis S. Gruber, Chicago, IL (US)

(21) Appl. No.: 18/611,415

(22) Filed: Mar. 20, 2024

Related U.S. Application Data
(63) Continuation of application No. 16/092,743, filed on Oct. 10, 2018, now Pat. No. 11,958,900.
(60) Provisional application No. 62/323,471, filed on Apr. 15, 2016.

(51) Int. Cl. C07K 16/24; A61K 35/17; A61K 38/00; A61P 25/28; C07K 16/44

(52) U.S. Cl. CPC C07K 16/24; A61K 35/17; A61P 25/28; C07K 16/44; A61K 38/00; C07K 2317/24; C07K 2317/92

(57) ABSTRACT

A method of treating a neurodegenerative disorder or MD comprises administering to a subject a composition comprising an AGE antibody.

### SEQUENCE LISTING INCORPORATION BY REFERENCE

A Sequence Listing XML file is submitted herewith. This file named “SIWO-011-CON-US_Sequence_Listing”, created on Apr. 24, 2025, having a file size of 65 KB is hereby incorporated by reference.

### BACKGROUND

Advanced glycation end-products (AGEs; also referred to AGE-modified proteins, or glycation end-products) arise from a non-enzymatic reaction of sugars with protein side-chains in aging cells (Ando, K. et al., Membrane Proteins of Human Erythrocytes Are Modified by Advanced Glycation End Products during Aging in the Circulation, Biochem Biophys Res Commun., Vol. 258, 123, 125 (1999)). This process begins with a reversible reaction between the reducing sugar and the amino group to form a Schiff base, which proceeds to form a covalently-bonded Amadori rearrangement product. Once formed, the Amadori product undergoes further rearrangement to produce AGEs. Hyperglycemia, caused by diabetes mellitus (DM), and oxidative stress promote this post-translational modification of membrane proteins (Lindsey J B, et al., “Receptor For Advanced Glycation End-Products (RAGE) and soluble RAGE (sRAGE): Cardiovascular Implications,” Diabetes Vascular Disease Research, Vol. 6(1), 7-14, (2009)). AGEs have been associated with several pathological conditions including diabetic complications, inflammation, retinopathy, nephropathy, atherosclerosis, stroke, endothelial cell dysfunction, and neurodegenerative disorders (Bierhaus A, “AGEs and their interaction with AGE-receptors in vascular disease and diabetes mellitus. I. The AGE concept,” Cardiovasc Res, Vol. 37(3), 586-600 (1998)).

Senescent cells are cells that are partially-functional or non-functional and are in a state of irreversible proliferative arrest. Senescence is a distinct state of a cell, and is associated with biomarkers, such as activation of the biomarker p16^{Ink4a}, and expression of β-galactosidase. Senescent cells are also associated with secretion of many factors involved in intercellular signaling, including pro-inflammatory factors; secretion of these factors has been termed the senescence-associated secretory phenotype, or SASP.

AGE-modified proteins are also a marker of senescent cells. This association between glycation end-products and senescence is well known in the art. See, for example, Gruber, L. (WO 2009/143411, 26 Nov. 2009), Ando, K. et al. (Membrane Proteins of Human Erythrocytes Are Modified by Advanced Glycation End Products during Aging in the Circulation, Biochem Biophys Res Commun., Vol. 258, 123, 125 (1999)), Ahmed, E. K. et al. (“Protein Modification and Replicative Senescence of WI-38 Human Embryonic Fibroblasts” Aging Cells, vol. 9, 252, 260 (2010)), Vlassara, H. et al. (Advanced Glycosylation Endproducts on Erythrocyte Cell Surface Induce Receptor-Mediated Phagocytosis by Macrophages, J. Exp. Med., Vol. 166, 539, 545 (1987)) and Vlassara et al. (“High-affinity-receptor-mediated Uptake and Degradation of Glucose-modified Proteins: A Potential Mechanism for the Removal of Senescent Macromolecules” Proc. Natl. Acad. Sci. USA 1, Vol. 82, 5588, 5591 (1985)). Furthermore, Ahmed, E. K. et al. indicates that glycation end-products are “one of the major causes of spontaneous damage to cellular and extracellular proteins” (Ahmed, E. K. et al., see above, page 353). Accordingly, the accumulation of glycation end-products is associated with senescence and lack of function.

A recent study has identified a causal link between cellular senescence and age-related disorders, such as sarcopenia. A research team at the Mayo Clinic in Rochester, Minnesota, demonstrated that effects of aging in mice could be delayed by eliminating senescent cells in their fat and muscle tissues without overt side effects (Baker, D. J. et al., “Clearance of p16^{Ink4a}-positive senescent cells delays ageing-associated disorders”, Nature, Vol. 479, pp. 232-236, (2011)). Elimination of senescent cells in transgenic mice was shown to substantially delay the onset of sarcopenia and cataracts, and to reduce senescence indicators in skeletal muscle and the eye. The study established that life-long and late-life treatment of transgenic mice for removal of senescent cells has no negative side effects and selectively delays age-related phenotypes that depend on cells (ld., page 234, col. 2, line 16 through page 235, col. 1, line 2). The authors theorized that removal of senescent cells may represent an avenue for treating or delaying age-related diseases in humans and improving healthy human lifespan (Id., page 235, col. 2, lines 38-51).

Neurodegenerative disorders are associated with abnormal cellular senescence in the central nervous system. Abnormal accumulation of senescent astrocytes has been associated with Alzheimer's disease (AD) (Bhat, R. et al., “Astrocyte Senescence as a Component of Alzheimer's Disease”, PLOS ONE, Vol. 7(9), e45069, pp. 1-10 (September 2012)). Microglial cell senescence associated with normal aging is exacerbated by the presence of the amyloid plaques indicative of AD (Flanary, B. E. et al., “Evidence That Aging And Amyloid Promote Microglial Cell Senescence”, Rejuvenation Research, Vol. 10(1), pp. 61-74 (March 2007)). The presence of AGEs with astrocytes and microglial cells in AD is further evidence of the presence of senescent cells (Takeda, A., et al. “Advanced glycation end products co-localize with astrocyes and microglial cells in Alzheimer's disease brain”, Acta Neuropathologica, Vol. 95, pp. 555-558 (1998)). On the basis of recently reported findings, Chinta et al. proposed that environmental stressors associated with Parkinson's disease (PD) may act in part by eliciting senescence within non-neuronal glial cells, contributing to the characteristic decline in neuronal integrity that occurs in this disorder (Chinta, S. J. et al. “Environmental stress, ageing and glial cell senescence: a novel mechanistic link to Parkinson's disease?”, J Intern Med, Vol. 273, pp. 429-436 (2013)). Astrocyte senescence is also associated with PD (M. Mori, “The Parkinsonian Brain: Cellular Senescence and Neurodegeneration, SAGE (Jun. 30, 2015) (sage.buckinstitute.org/the-parkinsonian-brain-cellular-senescence-and-neurodegeneration/). In a rodent model of familial amyotrophic lateral sclerosis (ALS) overexpressing mutant superoxide dismutase-1 (m-SOD1), the rate of astrocytes acquiring a senescent phenotype is accelerated (Das, M. M. and Svendsen, C. N., “Astrocytes show reduced support of motor neurons with aging that is accelerated in a rodent model of ALS”, Neurobiology of Aging, Vol. 36, pp. 1130-1139 (2015)). Even in multiple sclerosis (MS), microglia and macrophages are shifted toward a strongly proinflammatory phenotype, reminiscent of SASP, and may potentiate neuronal damage by releasing proinflammatory cytokines and molecules (Luessi, F., et al. “Neurodegeneration in multiple sclerosis: novel treatment strategies” Expert Rev. Neurother., Vol 9, pp. 1061-1077 (2012)).

Glial cells, such as astrocytes and microglial cells, provide support for normal brain functions. Astrocytes, also known collectively as astroglia, are star-shaped glial cells found in the brain and spinal cord. Astrocytes perform many functions, such as providing nutrients to nervous tissue, maintaining ion balance in extracellular fluids, and biochemical support of the cells that form the blood-brain barrier. Microglial cells act as macrophages in the brain and spinal cord. Microglial cells scavenge plaques, damaged neurons and infectious agents from the brain and spinal cord.

Some neurodegenerative disorders are also associated with abnormal cellular senescence outside the central nervous system. Most satellite cells, also known as myosatellite cells, present in the muscle tissue of ALS patients exhibit an abnormal senescent-like morphology, although they may be capable of proliferating in vitro (Pradat, P.-F. et al., “Abnormalities of satellite cells function in amyotrophic lateral sclerosis” Amyotrophic Lateral Sclerosis, Vol. 12, pp. 264-271 (2011)). Satellite cells are small multipotent cells found in mature muscle, which are able to give rise to additional satellite cells, or differentiate into myoblasts as well as provide additional myonuclei. In an animal model of Duchenne muscular dystrophy (MD), reduced proliferative capacity and premature senescence of myoblasts was observed (Wright, W. E., “Myoblast Senescence in Muscular Dystrophy” Exp Cell Res, Vol. 157, pp. 343-354 (1985)). Myoblasts are precursor cells which differentiate into myocytes (also referred to as muscle cells).

Neurodegenerative disorders are also associated with abnormal protein accumulations (King, O. D., et al., “The tip of the iceberg: RNA-binding proteins with prion-like domains in neurodegenerative disease” Brain Res. Vol. 1462, pp. 61-80 (2012)). A characteristic of PD and Lewy body dementia is the formation of Lewy bodies that form inside nerve cells. The primary structural component of the Lewy bodies is alpha-synuclein protein, in the form of fibrils. The presence of tangles and plaques are a characteristic of AD, the presence of which is used to definitively diagnose the condition. Plaques, composed of beta-amyloid protein (also referred to as amyloid beta, Ap or Abeta), accumulate between nerve cells. Tangles, composed of tau protein, form twisted fibers within cells. Prion diseases (also known as transmissible spongiform encephalopathies (TSEs)), include a variety of human and animal disorder such as Creutzfeldt-Jakob disease, variant Creutzfeldt-Jakob disease, bovine spongiform encephalopathy (“mad cow” disease), scrapie (in sheep and goats), chronic wasting disease (in deer and elk), kuru and fatal familial insomnia. Prion protein is a misfolded protein molecule which may propagate by transmitting a misfolded protein state, resulting in the accumulation of the misfolded protein and causing tissue damage and cell death (Dobson, D. M., “The structural basis of protein folding and its links with human disease” Phil. Trans. R. Soc. Lond. B, Vol. 356, pp. 133-145 (2001)). In these diseases, it is believed the protein is a normal protein which misfolds or forms an abnormal aggregate. In the case of some patients with familial ALS, a mutated superoxide dismutase-1 (SOD1) forms inclusions and accumulates (Kato, S., et al. “Advanced glycation endproduct-modified superoxide dismutase-1 (SOD1)-positive inclusions are common to familial amyotrophic lateral sclerosis patients with SOD1 gene mutations and transgenic mice expressing human SOD1 with a G85R mutation” Acta Neuropathol, Vol. 100, pp. 490-505 (2000)).

In some cases, the proteins are believed to directly cause the death of cells, while in others the protein is believed to cause inflammation indirectly causing death of cells. The inflammation is also believed to induce senescence in cells, which in turn further exacerbates inflammation due to the SASP, leading to a positive feedback advancing neurodegeneration (Golde, T. E., et al. “Proteinopathy-induced neuronal senescence: a hypothesis for brain failure in Alzheimer's and other neurodegenerative diseases” Alzheimer's Research & Therapy, Vol. 1, No. 5 (13 Oct. 2009)). Spreading of these inflammation-inducing proteins may also be exacerbated by senescent cells, through intercellular protein transfer (Biran, A., et al. “Senescent cells communicate via intercellular protein transfer” Genes & Development, Vol. 29, pp. 791-802 (2015)).

Immunotherapy for neurodegenerative disorders, using antibodies to neurodegenerative proteins associated with the neurodegenerative disorders, is showing some promise. Even when the antibodies are administered peripherally (that is, not into the CNS), positive effects have been observed.

### SUMMARY

In a first aspect, the present invention is a method of treating a neurodegenerative disorder or MD comprising administering to a subject a composition comprising an AGE antibody.

In a second aspect, the present invention is a method of killing senescent glial cells comprising administering to a subject a composition comprising an AGE antibody.

In a third aspect, the present invention is a method of killing senescent myoblasts and/or senescent myosatellite cells comprising administering to a subject a composition comprising an AGE antibody.

In a fourth aspect, the present invention is a method of treating a subject with a neurodegenerative disorder or MD comprising a first administering of an AGE antibody; followed by testing the subject for effectiveness of the first administration at treating the neurodegenerative disorder or MD; followed by a second administering of the AGE antibody.

In a fifth aspect, the present invention is a method of treating a neurodegenerative disorder or MD comprising killing or inducing apoptosis in senescent glial cells, senescent myoblasts and/or senescent myosatellite cells.

In a sixth aspect, the present invention is a composition for treating a neurodegenerative disorder comprising (i) an AGE antibody and (ii) serum, immune system cells, or both.

### Definitions

The term “neurodegenerative disorder” means disorders which result in neurons losing function and/or dying, in the central nervous system including the brain. Such disorders included central nervous system neurodegenerative disorders such as AD, PD, Lewy body dementia, MS, prion diseases (also known as transmissible spongiform encephalopathies (TSEs), including Creutzfeldt-Jakob disease, variant Creutzfeldt-Jakob disease, bovine spongiform encephalopathy (“mad cow” disease), scrapie (in sheep and goats), chronic wasting disease (in deer and elk), kuru and fatal familial insomnia), and ALS.

The terms “advanced glycation end-product,” “AGE,” “AGE-modified protein or peptide,” “glycation end-product” and “AGE antigen” refer to modified proteins or peptides that are formed as the result of the reaction of sugars with protein side chains that further rearrange and form irreversible cross-links. This process begins with a reversible reaction between a reducing sugar and an amino group to form a Schiff base, which proceeds to form a covalently-bonded Amadori rearrangement product. Once formed, the Amadori product undergoes further rearrangement to produce AGEs. AGE-modified proteins and antibodies to AGE-modified proteins are described in U.S. Pat. No. 5,702,704 to Bucala (“Bucala”) and U.S. Pat. No. 6,380,165 to Al-Abed et al. (“Al-Abed”). Glycated proteins or peptides that have not undergone the necessary rearrangement to form AGEs, such as N-deoxyfructosyllysine found on glycated albumin, are not AGEs. AGEs may be identified by the presence of AGE modifications (also referred to as AGE epitopes or AGE moieties) such as 2-(2-furoyl)-4(5)-(2-furanyl)-1H-imidazole (“FFI”); 5-hydroxymethyl-1-alkylpyrrole-2-carbaldehyde (“Pyrraline”); 1-alkyl-2-formyl-3,4-diglycosyl pyrrole (“AFGP”), a non-fluorescent model AGE; carboxymethyllysine; and pentosidine. ALI, another AGE, is described in Al-Abed.

“Neurodegenerative proteins” are proteins which accumulate in a patient having a neurodegenerative disorders and which are associated with the neurodegenerative disorder. Examples include, beta-amyloid protein plaques (associated with AD), tau protein tangles (associated with AD), mutated superoxide dismutase-1 (associated with ALS), prion protein aggregates (associated with TSEs) and alpha-synuclein protein fibrils (associated with PD and Lewy Body dementia). A “neurodegenerative protein” is the form of the protein which accumulates during the neurodegenerative disorder, typically a mutant or mis-folded form.

“An antibody that binds to an AGE-modified protein on a cell”, “anti-AGE antibody” or “AGE antibody” means an antibody or other protein that binds to an AGE-modified protein or peptide and includes a constant region of an antibody, where the protein or peptide which has been AGE-modified is a protein or peptide normally found bound on the surface of a cell, preferably a mammalian cell, more preferably a human, cat, dog, horse, camelid (for example, camel or alpaca), cattle, sheep, or goat cell. “An antibody that binds to an AGE-modified protein on a cell”, “anti-AGE antibody” or “AGE antibody” does not include an antibody or other protein which binds with the same specificity and selectivity to both the AGE-modified protein or peptide, and the same non-AGE-modified protein or peptide (that is, the presence of the AGE modification does not increase binding). AGE-modified albumin is not an AGE-modified protein on a cell, because albumin is not a protein normally found bound on the surface of cells. “An antibody that binds to an AGE-modified protein on a cell”, “anti-AGE antibody” or “AGE antibody” only includes those antibodies which lead to removal, destruction, or death of the cell. Also included are antibodies which are conjugated, for example to a toxin, drug, or other chemical or particle. Preferably, the antibodies are monoclonal antibodies, but polyclonal antibodies are also possible.

The term “senescent cell” means a cell which is in a state of irreversible proliferative arrest and expresses one or more biomarkers of senescence, such as activation of p16^{Ink4a} or expression of β-galactosidase. Also included are cells which express one or more biomarkers of senescence, do not proliferate in vivo, but may proliferate in vitro under certain conditions, such as some satellite cells found in the muscles of ALS patients.

The term “variant” means a nucleotide, protein or amino acid sequence different from the specifically identified sequences, wherein one or more nucleotides, proteins or amino acid residues is deleted, substituted or added. Variants may be naturally-occurring allelic variants, or non-naturally-occurring variants. Variants of the identified sequences may retain some or all of the functional characteristics of the identified sequences.

The term “percent (%) sequence identity” is defined as the percentage of amino acid residues in a candidate sequence that are identical to the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using publicly available computer software such as BLAST, BLAST-2, ALIGN or Megalign (DNASTAR) software. Preferably, % sequence identity values are generated using the sequence comparison computer program ALIGN-2. The ALIGN-2 sequence comparison computer program is publicly available from Genentech, Inc. (South San Francisco, CA), or may be compiled from the source code, which has been filed with user documentation in the U.S. Copyright Office and is registered under U.S. Copyright Registration No. TXU510087. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained using the ALIGN-2 computer program.

### BRIEF DESCRIPTION OF THE DRAWING

FIG. 1 is a graph of the response versus time in an antibody binding experiment.

FIG. 2A is a photograph of cells of an Alzheimer's disease sample showing carboxymethyllysine stained gray and phosphorylated tau stained light gray.

FIG. 2B is a photograph of cells of an Alzheimer's disease sample showing carboxymethyllysine stained gray and amyloid precursor protein stained light gray.

FIG. 2C is a photograph of cells of a Parkinson's disease sample from the substantia nigra showing carboxymethyllysine stained gray and alpha synuclein stained light gray.

FIG. 2D is a photograph of cells of a Parkinson's disease sample from the ventral tegmental area showing carboxymethyllysine stained gray and alpha synuclein stained light gray.

### DETAILED DESCRIPTION

The present invention makes use of antibodies that bind to an AGE-modified protein on a cell, to remove or kill senescent glial cells, such as senescent astrocytes, and senescent microglial cells, to treat neurodegenerative disorders such as AD, PD, Lewy body dementia, MS, prion diseases (also known as transmissible spongiform encephalopathies (TSEs), including Creutzfeldt-Jakob disease, variant Creutzfeldt-Jakob disease, bovine spongiform encephalopathy (“mad cow” disease), scrapie (in sheep and goats), chronic wasting disease (in deer and elk), kuru and fatal familial insomnia), and ALS. Preferably, the antibodies are administered into the central nervous system to most efficiently remove these senescent cells; however, peripheral administration (that is, not into the central nervous system but into the peripheral circulatory system) is also effective, since the astrocytes help form the blood-brain barrier. Stem cell present in the patient's central nervous system will then grow and expand to replace cells which were removed. Alternatively, autologous transplantation of the patient's own stem cells, or transplantation of donor stem cells (which may be expanded ex vivo) may also be used to replace cells which were removed.

The present invention also makes use of antibodies that bind to an AGE-modified protein on a cell, to remove or kill senescent glial cells and/or senescent myosatellite cells, to treat ALS. Preferably, the antibodies are administered into the peripheral circulation (such as traditional intravenous administration) to most efficiently remove these senescent cells. The antibodies may also be administered intramuscularly, where the senescent myosatellite cells are found. Stem cell present in the patient's muscles will then grow and expand to replace cells which were removed. Alternatively, autologous transplantation of the patient's own stem cells, or transplantation of donor stem cells (which may be expanded ex vivo) may also be used to replace cells which were removed.

The present invention also makes use of antibodies that bind to an AGE-modified protein on a cell, to remove or kill senescent myoblasts and/or senescent myosatellite cells, to treat MD and ALS. Preferably, the antibodies are administered into the peripheral circulation (such as traditional intravenous administration) to most efficiently remove these senescent cells. The antibodies may also be administered intramuscularly, where the senescent myoblasts and myosatellite cells are found. Stem cell present in the patient's muscles will then grow and expand to replace cells which were removed. Alternatively, autologous transplantation of the patient's own stem cells, or transplantation of donor stem cells (which may be expanded ex vivo) may also be used to replace cells which were removed. See, for example, Rouger et al. “Systemic Delivery of Allogenic Muscle Stem Cells Induces Long-Term Muscle Repair and Clinical Efficacy in Duchenne Muscular Dystrophy Dogs” The American Journal of Pathology, Vol. 179, No. 5, 2501-2518 (November 2011).

Senescence begins with damage or stress (such as overstimulation by growth factors) of cells. The damage or stress negatively impacts mitochondrial DNA in the cells to cause them to produce free radicals which react with sugars in the cell to form methyl glyoxal (MG). MG in turn reacts with proteins or lipids to generate advanced glycation end products (AGEs). In the case of the protein component lysine, glyoxal reacts to form carboxymethyllysine, which is an AGE. AGEs also form from non-enzymatic reaction of sugars in the blood with external cell proteins.

Damage or stress to mitochondrial DNA also sets off a DNA damage response which induces the cell to produce cell cycle blocking proteins. These blocking proteins prevent the cell from dividing. Continued damage or stress causes (1) mTOR production, which in turn activates protein synthesis and inactivates protein breakdown, and (2) an SASP (senescence associated secretory phenotype) wherein growth stimulatory and inhibitory factors are secreted to cause senescence in other cells (the senescent cell bystander effect). Further stimulation of the cells leads to programmed cell death (apoptosis).

An antibody that binds to an AGE-modified protein on a cell (“anti-AGE antibody” or “AGE antibody”) is known in the art. Examples include those described in U.S. Pat. No. 5,702,704 (Bucala) and U.S. Pat. No. 6,380,165 (Al-Abed et al.). Examples include an antibody that binds to one or more AGE-modified proteins having an AGE modification such as FFI, pyrraline, AFGP, ALI, carboxymethyllysine, carboxyethyllysine and pentosidine, and mixtures of such antibodies. Preferably, the antibody binds carboxymethyllysine-modified proteins. Preferably, the antibody is non-immunogenic to the animal in which it will be used, such as non-immunogenic to humans; companion animals including cats, dogs and horses; and commercially important animals, such camels (or alpaca), cattle (bovine), sheep, and goats. More preferably, the antibody has the same species constant region as antibodies of the animal to reduce the immune response against the antibody, such as being humanized (for humans), felinized (for cats), caninized (for dogs), equuinized (for horses), camelized (for camels or alpaca), bovinized (for cattle), ovinized (for sheep), or caperized (for goats). Most preferably, the antibody is identical to that of the animal in which it will be used (except for the variable region), such as a human antibody, a cat antibody, a dog antibody, a horse antibody, a camel antibody, a bovine antibody, a sheep antibody or a goat antibody. Details of the constant regions and other parts of antibodies for these animals are described below. Preferably, the antibody is a monoclonal antibody.

A particularly preferred AGE antibody is an antibody which binds to a protein or peptide that exhibits a carboxymethyllysine modification. Carboxymethyllysine (also known as CML, N(epsilon)-(carboxymethyl)lysine, N(6)-carboxymethyllysine, or 2-Amino-6-(carboxymethylamino)hexanoic acid) is found on proteins or peptides and lipids as a result of oxidative stress and chemical glycation, and has been correlated with aging. CML-modified proteins or peptides are recognized by the receptor RAGE which is expressed on a variety of cells. CML has been well-studied and CML-related products are commercially available. For example, Cell Biolabs, Inc. sells CML-BSA antigens, CML polyclonal antibodies, CML immunoblot kits, and CML competitive ELISA kits (www.cellbiolabs.com/cml-assays). A particularly preferred antibody includes the variable region of the commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, MN; catalog no. MAB3247), modified to have a human constant region (or the constant region of the animal into which it will be administered). Commercially-available antibodies, such as the carboxymethyl lysine antibody corresponding to catalog no. MAB3247 from R&D Systems, Inc., may be intended for diagnostic purposes and may contain material that is not suited for use in animals or humans. Preferably, commercially-available antibodies are purified and/or isolated prior to use in animals or humans to remove toxins or other potentially-harmful material.

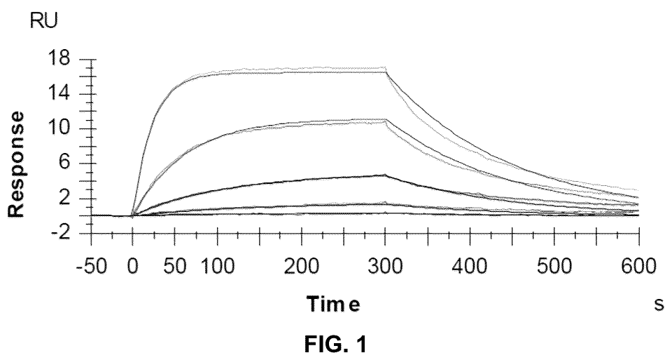

The AGE antibody has low rate of dissociation from the antibody-antigen complex, or k_{d} (also referred to as k_{back} or off-rate), preferably at most 9×10⁻³, 8×10⁻³, 7×10⁻³ or 6×10⁻³ (sec⁻¹). The AGE antibody has a high affinity for the AGE-modified protein of a cell, which may be expressed as a low dissociation constant K_{D} of at most 9×10⁻⁶, 8×10⁻⁶, 7×10⁻⁶, 6×10⁻⁶, 5×10⁻⁶, 4×10⁻⁶ or 3×10⁻⁶ (M). Preferably, the binding properties of the AGE antibody is greater than, similar to, or the same as, the carboxymethyl lysine MAb (Clone 318003) available from R&D Systems, Inc. (Minneapolis, MN; catalog no. MAB3247), illustrated in FIG. 1.

The anti-AGE antibody may destroy AGE-modified cells through antibody-dependent cell-mediated cytotoxicity (ADCC). ADCC is a mechanism of cell-mediated immune defense in which an effector cell of the immune system actively lyses a target cell whose membrane-surface antigens have been bound by specific antibodies. ADCC may be mediated by natural killer (NK) cells, macrophages, neutrophils or eosinophils. The effector cells bind to the Fc portion of the bound antibody.

The AGE antibody may be conjugated to an agent that causes the destruction of AGE-modified cells. Such agents may be a toxin, a cytotoxic agent, magnetic nanoparticles, and magnetic spin-vortex discs.

A toxin, such as pore-forming toxins (PFT) (Aroian R. et al., “Pore-Forming Toxins and Cellular Non-Immune Defenses (CNIDs),” Current Opinion in Microbiology, 10:57-61 (2007)), conjugated to an AGE antibody may be injected into a patient to selectively target and remove AGE-modified cells. The AGE antibody recognizes and binds to AGE-modified cells. Then, the toxin causes pore formation at the cell surface and subsequent cell removal through osmotic lysis.

Magnetic nanoparticles conjugated to the AGE antibody may be injected into a patient to target and remove AGE-modified cells. The magnetic nanoparticles can be heated by applying a magnetic field in order to selectively remove the AGE-modified cells.

As an alternative, magnetic spin-vortex discs, which are magnetized only when a magnetic field is applied to avoid self-aggregation that can block blood vessels, begin to spin when a magnetic field is applied, causing membrane disruption of target cells. Magnetic spin-vortex discs, conjugated to AGE antibodies specifically target AGE-modified cell types, without removing other cells.

Antibodies typically comprise two heavy chains and two light chains of polypeptides joined to form a “Y” shaped molecule. The constant region determines the mechanism used to target the antigen. The amino acid sequence in the tips of the “Y” (the variable region) varies among different antibodies. This variation gives the antibody its specificity for binding antigen. The variable region, which includes the ends of the light and heavy chains, is further subdivided into hypervariable (HV—also sometimes referred to as complementarity determining regions, or CDRs) and framework (FR) regions. When antibodies are prepared recombinantly, it is also possible to have a single antibody with variable regions (or complementary determining regions) that bind to two different antigens, with each tip of the “Y” being specific to each antigen; these are referred to as bi-specific antibodies.

A humanized anti-AGE antibody according to the present invention may have the human constant region sequence of amino acids shown in SEQ ID NO: 22. The heavy chain complementarity determining regions of the humanized anti-AGE antibody may have one or more of the protein sequences shown in SEQ ID NO: 23 (CDR1H), SEQ ID NO: 24 (CDR2H) and SEQ ID NO: 25 (CDR3H). The light chain complementarity determining regions of the humanized anti-AGE antibody may have one or more of the protein sequences shown in SEQ ID NO: 26 (CDR1 L), SEQ ID NO: 27 (CDR2L) and SEQ ID NO: 28 (CDR3L).

The heavy chain of human (Homo sapiens) antibody immunoglobulin G1 may have or may include the protein sequence of SEQ ID NO: 1. The variable domain of the heavy chain may have or may include the protein sequence of SEQ ID NO: 2. The kappa light chain of human (Homo sapiens) antibody immunoglobulin G1 may have or may include the protein sequence of SEQ ID NO: 3. The variable domain of the kappa light chain may have or may include the protein sequence of SEQ ID NO: 4. The variable regions may be codon-optimized, synthesized and cloned into expression vectors containing human immunoglobulin G1 constant regions. In addition, the variable regions may be used in the humanization of non-human antibodies.

The antibody heavy chain may be encoded by the DNA sequence of SEQ ID NO: 12, a murine anti-AGE immunoglobulin G2b heavy chain. The protein sequence of the murine anti-AGE immunoglobulin G2b heavy chain encoded by SEQ ID NO: 12 is shown in SEQ ID NO: 16. The variable region of the murine antibody is shown in SEQ ID NO: 20, which corresponds to positions 25-142 of SEQ ID NO: 16. The antibody heavy chain may alternatively be encoded by the DNA sequence of SEQ ID NO: 13, a chimeric anti-AGE human immunoglobulin G1 heavy chain. The protein sequence of the chimeric anti-AGE human immunoglobulin G1 heavy chain encoded by SEQ ID NO: 13 is shown in SEQ ID NO: 17. The chimeric anti-AGE human immunoglobulin includes the murine variable region of SEQ ID NO: 20 in positions 25-142. The antibody light chain may be encoded by the DNA sequence of SEQ ID NO: 14, a murine anti-AGE kappa light chain. The protein sequence of the murine anti-AGE kappa light chain encoded by SEQ ID NO: 14 is shown in SEQ ID NO: 18. The variable region of the murine antibody is shown in SEQ ID NO: 21, which corresponds to positions 21-132 of SEQ ID NO: 18. The antibody light chain may alternatively be encoded by the DNA sequence of SEQ ID NO: 15, a chimeric anti-AGE human kappa light chain. The protein sequence of the chimeric anti-AGE human kappa light chain encoded by SEQ ID NO: 15 is shown in SEQ ID NO: 19. The chimeric anti-AGE human immunoglobulin includes the murine variable region of SEQ ID NO: 21 in positions 21-132.

A humanized anti-AGE antibody according to the present invention may have or may include one or more humanized heavy chains or humanized light chains. A humanized heavy chain may be encoded by the DNA sequence of SEQ ID NO: 30, 32 or 34. The protein sequences of the humanized heavy chains encoded by SEQ ID NOs: 30, 32 and 34 are shown in SEQ ID NOs: 29, 31 and 33, respectively. A humanized light chain may be encoded by the DNA sequence of SEQ ID NO: 36, 38 or 40. The protein sequences of the humanized light chains encoded by SEQ ID NOs: 36, 38 and 40 are shown in SEQ ID NOs: 35, 37 and 39, respectively. Preferably, the humanized anti-AGE antibody maximizes the amount of human sequence while retaining the original antibody specificity. A complete humanized antibody may be constructed that contains a heavy chain having a protein sequence chosen from SEQ ID NOs: 29, 31 and 33 and a light chain having a protein sequence chosen from SEQ ID NOs: 35, 37 and 39.

The protein sequence of an antibody from a non-human species may be modified to include the variable domain of the heavy chain having the sequence shown in SEQ ID NO: 2 or the kappa light chain having the sequence shown in SEQ ID NO: 4. The non-human species may be a companion animal, such as the domestic cat or domestic dog, or livestock, such as cattle, the horse or the camel. Preferably, the non-human species is not the mouse. The heavy chain of the horse (Equus caballus) antibody immunoglobulin gamma 4 may have or may include the protein sequence of SEQ ID NO: 5 (EMBL/GenBank accession number AY445518). The heavy chain of the horse (Equus caballus) antibody immunoglobulin delta may have or may include the protein sequence of SEQ ID NO: 6 (EMBL/GenBank accession number AY631942). The heavy chain of the dog (Canis familiaris) antibody immunoglobulin A may have or may include the protein sequence of SEQ ID NO: 7 (GenBank accession number L36871). The heavy chain of the dog (Canis familiaris) antibody immunoglobulin E may have or may include the protein sequence of SEQ ID NO: 8 (GenBank accession number L36872). The heavy chain of the cat (Felis catus) antibody immunoglobulin G2 may have or may include the protein sequence of SEQ ID NO: 9 (DDBJ/EMBL/GenBank accession number KF811175).

Animals of the camelid family, such as camels (Camelus dromedarius and Camelus bactrianus), llamas (Lama glama, Lama pacos and Lama vicugna), alpacas (Vicugna pacos) and guanacos (Lama guanicoe), have a unique antibody that is not found in other mammals. In addition to conventional immunoglobulin G antibodies composed of heavy and light chain tetramers, camelids also have heavy chain immunoglobulin G antibodies that do not contain light chains and exist as heavy chain dimers. These antibodies are known as heavy chain antibodies, HCAbs, single-domain antibodies or sdAbs, and the variable domain of a camelid heavy chain antibody is known as the VHH. The camelid heavy chain antibodies lack the heavy chain CH1 domain and have a hinge region that is not found in other species. The variable region of the Arabian camel (Camelus dromedarius) single-domain antibody may have or may include the protein sequence of SEQ ID NO: 10 (GenBank accession number AJ245148). The variable region of the heavy chain of the Arabian camel (Camelus dromedarius) tetrameric immunoglobulin may have or may include the protein sequence of SEQ ID NO: 11 (GenBank accession number AJ245184).

In addition to camelids, heavy chain antibodies are also found in cartilaginous fishes, such as sharks, skates and rays. This type of antibody is known as an immunoglobulin new antigen receptor or IgNAR, and the variable domain of an IgNAR is known as the VNAR. The IgNAR exists as two identical heavy chain dimers composed of one variable domain and five constant domains each. Like camelids, there is no light chain.

The protein sequences of additional non-human species may be readily found in online databases, such as the International ImMunoGeneTics Information System (www.imgt.org), the European Bioinformatics Institute (www.ebi.ac.uk), the DNA Databank of Japan (ddbj.nig.ac.jp/arsa) or the National Center for Biotechnology Information (www.ncbi.nlm.nih.gov).

Additional DNA and protein sequences may be found in U.S. Provisional Patent Application No. 62/485,246, which is herein incorporated by reference.

An anti-AGE antibody or a variant thereof may include a heavy chain variable region having at least 90%, 91%, 92%, 93%, 94%, 95%, 96%, 97%, 98%, 99%, or 100% sequence identity to the amino acid sequence of SEQ ID NO: 2 or SEQ ID NO: 20, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain variable region having at least 90%, 91%, 92%, 93%, 94%, 95%, 96%, 97%, 98%, 99%, or 100% sequence identity to the amino acid sequence of SEQ ID NO: 4 or SEQ ID NO: 21, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

Alternatively, the antibody may have the complementarity determining regions of commercially available mouse anti-glycation end-product antibody raised against carboxymethyl lysine conjugated with keyhole limpet hemocyanin (CML-KLH), the carboxymethyl lysine MAb (Clone 318003) available from R&D Systems, Inc. (Minneapolis, MN; catalog no. MAB3247).

The antibody may have or may include constant regions which permit destruction of targeted cells by a subject's immune system. Particularly preferred is a monoclonal antibody specific for carboxymethyllysine which is the AGE most commonly found in humans. Preferably, such an antibody includes a complement binding portion (Fc) which stimulates an increase in system natural killer (NK) cell Fc receptors (1) causing the NK cells to bind to the antibody, which in turn, has bound to senescent cells, and (2) initiate a lytic reaction. This causes the senescent cells to undergo apoptosis and be broken up into fragments which are taken up by macrophages, broken down and cleared from the body.

Mixtures of antibodies that bind to more than one type AGE of AGE-modified proteins may also be used.

Bi-specific antibodies, which are AGE antibodies directed to two different epitopes, may also be used. Such antibodies will have a variable region (or complementary determining region) from those of one AGE antibody, and a variable region (or complementary determining region) from a different antibody.

Antibody fragments may be used in place of whole antibodies. For example, immunoglobulin G may be broken down into smaller fragments by digestion with enzymes. Papain digestion cleaves the N-terminal side of inter-heavy chain disulfide bridges to produce Fab fragments. Fab fragments include the light chain and one of the two N-terminal domains of the heavy chain (also known as the Fd fragment). Pepsin digestion cleaves the C-terminal side of the inter-heavy chain disulfide bridges to produce F(ab′)₂ fragments. F(ab′)₂ fragments include both light chains and the two N-terminal domains linked by disulfide bridges. Pepsin digestion may also form the Fv (fragment variable) and Fc (fragment crystallizable) fragments. The Fv fragment contains the two N-terminal variable domains. The Fc fragment contains the domains which interact with immunoglobulin receptors on cells and with the initial elements of the complement cascade. Pepsin may also cleave immunoglobulin G before the third constant domain of the heavy chain (C_{H}3) to produce a large fragment F(abc) and a small fragment pFc′. Single domain antibodies, which include a heavy chain CDR and are conjugated to a toxin or other moiety for causing cell death or destruction, may also be used, and are know to pass through the blood-brain barrier. Antibody fragments may alternatively be produced recombinantly.

If additional antibodies are desired, they can be produced using well-known methods. For example, polyclonal antibodies (pAbs) can be raised in a mammalian host by one or more injections of an immunogen, and if desired, an adjuvant. Typically, the immunogen (and adjuvant) is injected in a mammal by a subcutaneous or intraperitoneal injection. The immunogen may be an AGE-modified protein of a cell, such as AGE-antithrombin III, AGE-calmodulin, AGE-insulin, AGE-ceruloplasmin, AGE-collagen, AGE-cathepsin B, AGE-albumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-RNAse, AGE-hemoglobin such as AGE-human hemoglobin, AGE-albumin such as AGE-bovine serum albumin (AGE-BSA) and AGE-human serum albumin, AGE-low density lipoprotein (AGE-LDL) and AGE-collagen IV. AGE-modified cells, such as AGE-modified erythrocytes, whole, lysed, or partially digested, may also be used as AGE antigens. Examples of adjuvants include Freund's complete, monophosphoryl Lipid A synthetic-trehalose dicorynomycolate, aluminum hydroxide (alum), heat shock proteins HSP 70 or HSP96, squalene emulsion containing monophosphoryl lipid A, a2-macroglobulin and surface active substances, including oil emulsions, pleuronic polyols, polyanions and dinitrophenol. To improve the immune response, an immunogen may be conjugated to a polypeptide that is immunogenic in the host, such as keyhole limpet hemocyanin (KLH), serum albumin, bovine thyroglobulin, cholera toxin, labile enterotoxin, silica particles or soybean trypsin inhibitor. Alternatively, pAbs may be made in chickens, producing IgY molecules.

Monoclonal antibodies (mAbs) may also be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Other techniques may be used, such as the EBV-hybridoma technique. Techniques for the generation of chimeric antibodies by splicing genes encoding the variable domains of antibodies to genes of the constant domains of human (or other animal) immunoglobulin result in “chimeric antibodies” that are substantially human (humanized) or substantially “ized” to another animal (such as cat, dog, horse, camel or alpaca, cattle, sheep, or goat) at the amino acid level. If desired, the mAbs may be purified from the culture medium or ascites fluid by conventional procedures, such as protein A-sepharose, hydroxylapatite chromatography, gel electrophoresis, dialysis, ammonium sulfate precipitation or affinity chromatography. Additionally, human monoclonal antibodies can be generated by immunization of transgenic mice containing a third copy IgG human trans-loci and silenced endogenous mouse Ig loci or using human-transgenic mice. Production of humanized monoclonal antibodies and fragments thereof can also be generated through phage display technologies.

A “pharmaceutically acceptable carrier” includes any and all solvents, dispersion media, coatings, antibacterial and antifungal agents, isotonic and absorption delaying agents, and the like, compatible with pharmaceutical administration. Preferred examples of such carriers or diluents include water, saline, Ringer's solutions and dextrose solution. Supplementary active compounds can also be incorporated into the compositions. Solutions and suspensions used for parenteral administration can include a sterile diluent, such as water for injection, saline solution, polyethylene glycols, glycerin, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions or dispersions for the extemporaneous preparation of sterile injectable solutions or dispersion. Various excipients may be included in pharmaceutical compositions of antibodies suitable for injection. For administration by injection, suitable carriers include physiological saline, bacteriostatic water, CREMOPHOR EL® (BASF; Parsippany, NJ) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. Various antibacterial and anti-fungal agents, for example, parabens, chlorobutanol, phenol, ascorbic acid, and thimerosal, can contain microorganism contamination. Isotonic agents such as sugars, polyalcohols, such as manitol, sorbitol, and sodium chloride can be included in the composition. Compositions that can delay absorption include agents such as aluminum monostearate and gelatin. Sterile injectable solutions can be prepared by incorporating antibodies, and optionally other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

For administration by inhalation, the antibodies are delivered as an aerosol spray from a nebulizer or a pressurized container that contains a suitable propellant, for example, a gas such as carbon dioxide. Antibodies may also be delivered via inhalation as a dry powder, for example using the iSPERSE™ inhaled drug deliver platform (PULMATRIX, Lexington, Mass.). The use of AGE antibodies which are chicken antibodies (IgY) may be non-immunogenic in a variety of animals, including humans, when administered by inhalation.

An appropriate dosage level of each type of antibody will generally be about 0.01 to 500 mg per kg patient body weight. Preferably, the dosage level will be about 0.1 to about 250 mg/kg; more preferably about 0.5 to about 100 mg/kg. A suitable dosage level may be about 0.01 to 250 mg/kg, about 0.05 to 100 mg/kg, or about 0.1 to 50 mg/kg. Within this range the dosage may be 0.05 to 0.5, 0.5 to 5 or 5 to 50 mg/kg. Although each type of antibody may be administered on a regimen of 1 to 4 times per day, such as once or twice per day, antibodies typically have a long half-life in vivo. Accordingly, each type of antibody may be administered once a day, once a week, once every two or three weeks, once a month, or once every 60 to 90 days.

A subject that receives administration of an AGE antibody may be tested to determine if it has been effective to treat the neurodegenerative disorder, by measuring changes in neurological function or cognitive function, or by the increase or decrease in the presence of a neurodegenerative protein associated with the neurodegenerative disorder. In the case of most neurodegenerative disorders, tests to measure the presence, severity and/or progression of the neurodegenerative disorder are well known. Administration of antibody and subsequent testing may be repeated until the desired therapeutic result is achieved, for example by evaluating the patient for the neurodegenerative disorder or evaluating the patient if the senescent cells have been killed.

Unit dosage forms can be created to facilitate administration and dosage uniformity. Unit dosage form refers to physically discrete units suited as single dosages for the subject to be treated, containing a therapeutically effective quantity of one or more types of antibodies in association with the required pharmaceutical carrier. Preferably, the unit dosage form is in a sealed container and is sterile.

Any mammal that could develop neurodegenerative disorders may be treated by the methods herein described. Humans are a preferred mammal for treatment. Other mammals that may be treated include mice, rats, goats, sheep, cows, horses and companion animals, such as dogs or cats. A subject in need of treatment may be identified by the diagnosis of a neurodegenerative disorder.

In the case of central nervous system neurodegenerative disorders, it may be preferably to administer the composition containing the AGE antibody directly into the central nervous system. Examples of such administration include intrathecal administration; administration into the ventricular system of the brain (intraventricular administration), for example, through a catheter or a permanent shunt, or other administration device which may be placed during a ventriculostomy (see, for example, Takami, A. et al. “Treatment of primary central nervous system lymphoma with induction of complement-dependent cytotoxicity by intraventricular administration of autologous-serum-supplemented rituximab”, Cancer Sci. Vol. 97, pp. 80-83 (January 2006)); and administered by convection enhanced delivery (CED) (see, for example, Chen, K. S., et al. “MONOCLONAL ANTIBODY THERAPY FOR MALIGNANT GLIOMA” chapter 10 of Glioma: Immunotherapeutic Approaches, pp. 132-141 (ed. R. Yamanaka; Landes Bioscience and Springer Science+Business Media, 2012)). All such central nervous system administration may optionally also include administration of a serum supplement (such as autologous serum), to enhance the cell killing properties of the AGE antibody; administration of serum supplement may be prior to, simultaneous with, or subsequent to, the administration of the AGE antibody. Optionally, any of the composition containing AGE antibodies described herein may further contain a serum supplement (such as an autologous serum supplement). In place of a serum supplement, or in addition to a serum supplement, purified immune system cells may also be used, either autologous immune system cells, or immune system cells from a donor; examples of such cells include natural killer cells. In addition to, or instead of, the patient's or a donor's natural killer cells, artificial natural killer cells such as those of NANTKWEST®, engineered to bind directly to antibodies, or engineered to bind directly to an AGE antigen (such as carboxymethyllysine) (see www.nantkwest.com).

The anti-AGE antibodies may be used in cell separation processes, such as magnetic cell separation. In magnetic cell separation, the anti-AGE antibodies are attached to magnetic beads through a process called coating. The coated magnetic beads may then specifically bind to AGE-modified cells. The AGE-modified cells that have bound to anti-AGE antibodies coated on magnetic beads will then respond to an applied magnetic field, allowing the AGE-modified cells to be separated from non-AGE-modified cells. Magnetic cell separation may be used to isolate AGE-modified cells from tissue samples and fluid samples. The magnetic beads may be microbeads (0.5-500 μm) or nanoparticles (5-500 nm). Anti-AGE antibodies coated on magnetic beads may also be used in isolation processes such as immunoassays and immunoprecipitation. Similarly, anti-AGE antibodies coated on magnetic beads may be used to specifically target and separate AGE-modified proteins or peptides from tissue samples and fluid samples. The anti-AGE antibodies may be used in other cell separation processes such as flow cytometry and cell sorting.

The anti-AGE antibodies may be used in cellular purification processes, such as immunopanning and immunoadsorption. Purification processes are useful in isolating desirable or unwanted cells from tissue cultures, cell cultures or blood. Cellular purification may be used in transplantations, such as a bone marrow transplant, or transfusions, such as a blood transfusion. Cellular purification is especially useful in autologous stem cell transplantation during chemotherapy to remove metastasizing malignant cells and concentrate beneficial stem cells. Immunopanning or immunoadsorption using an anti-AGE antibody may isolate AGE-modified cells from a tissue culture, cell culture or blood sample.

The one-letter amino acid sequence that corresponds to SEQ ID NO: 1 is shown below:

```
                10 20 30 40
MNLLLILTFV AAAVAQVQLL QPGAELVKPG ASVKLACKAS
                50 60 70 80
GYLFTTYWMH WLKQRPGQGL EWIGEISPTN GRAYYNARFK
                90 100 110 120
SEATLTVDKS SNTAYMQLSS LTSEASAVYY CARAYGNYEF
              130 140 150 160
AYWGQGTLVT VSVASTKGPS VFPLAPSSKS TSGGTAALGC
              170 180 190 200
LVKDYFPEPV TVSWNSGALT SGVHTFPAVL QSSGLYSLSS
              210 220 230 240
VVTVPSSSLG TQTYICNVNH KPSNTKVDKK VEPKSCDKTH
              250 260 270 280
TCPPCPAPEL LGGPSVFLFP PKPKDTLMIS RTPEVTCVVV
              290 300 310 320
DVSHEDPEVK FNWYVDGVEV HNAKTKPREE QYNSTYRVVS
              330 340 350 360
VLTVLHQDWL NGKEYKCKVS NKALPAPIEK TISKAKGQPR
              370 380 390 400
EPQVYTLPPS REEMTKNQVS LTCLVKGFYP SDIAVEWESN
              410 420 430 440
GQPENNYKTT PPVLDSDGSF FLYSKLTVDK SRWQQGNVFS
              450 460
CSVMHEALHN HYTQKSLSLS PGK
```

Positions 16-133 of the above amino acid sequence correspond to SEQ ID NO: 2. Positions 46-50 of the above amino acid sequence correspond to SEQ ID NO: 41. Positions 65-81 of the above amino acid sequence correspond to SEQ ID NO: 42. Positions 114-122 of the above amino acid sequence correspond to SEQ ID NO: 43.

The one-letter amino acid sequence that corresponds to SEQ ID NO: 3 is shown below:

```
                10 20 30 40
MNLLLILTFV AAAVADVVMT QTPLSLPVSL GDQASISCRS
                50 60 70 80
RQSLVNSNGN TFLQWYLQKP GQSPKLLIYK VSLRFSGVPD
                90 100 110 120
RFSGSGSGTD FTLKISRVEA EDLGLYFCSQ STHVPPTFGG
              130 140 150 160
GTKLEIKRTV AAPSVFIFPP SDEQLKSGTA SVVCLLNNFY
              170 180 190 200
PREAKVQWKV DNALQSGNSQ ESVTEQDSKD STYSLSSTLT
              210 220 230
LSKADYEKHK VYACEVTHQG LSSPVTKSFN RGEC
```

Positions 16-128 of the above amino acid sequence correspond to SEQ ID NO: 4. Optionally, the arginine (Arg or R) residue at position 128 of SEQ ID NO: 4 may be omitted. Positions 39-54 of the above amino acid sequence correspond to SEQ ID NO: 44. Positions 70-76 of the above amino acid sequence correspond to SEQ ID NO: 45. Positions 109-117 of the above amino acid sequence correspond to SEQ ID NO: 46.

The DNA sequence that corresponds to SEQ ID NO: 12 is shown below:

ATGGACCCCAAGGGCAGCCTGAGCTGGAGAATCCTGCTGTTCCTGAGCCT
GGCCTTCGAGCTGAGCTACGGCCAGGTGCAGCTGCTGCAGCCAGGTGCCG
AGCTCGTGAAACCTGGCGCCTCTGTGAAGCTGGCCTGCAAGGCTTCCGGC
TACCTGTTCACCACCTACTGGATGCACTGGCTGAAGCAGAGGCCAGGCCA
GGGCCTGGAATGGATCGGCGAGATCTCCCCCACCAACGGCAGAGCCTACT
ACAACGCCCGGTTCAAGTCCGAGGCCACCCTGACCGTGGACAAGTCCTCC
AACACCGCCTACATGCAGCTGTCCTCCCTGACCTCTGAGGCCTCCGCCGT
GTACTACTGCGCCAGAGCTTACGGCAACTACGAGTTCGCCTACTGGGGCC
AGGGCACCCTCGTGACAGTGTCTGTGGCTAAGACCACCCCTCCCTCCGTG
TACCCTCTGGCTCCTGGCTGTGGCGACACCACCGGATCCTCTGTGACCCT
GGGCTGCCTCGTGAAGGGCTACTTCCCTGAGTCCGTGACCGTGACCTGGA
ACTCCGGCTCCCTGTCCTCCTCCGTGCACACCTTTCCAGCCCTGCTGCAG
TCCGGCCTGTACACCATGTCCTCCAGCGTGACAGTGCCCTCCTCCACCTG
GCCTTCCCAGACCGTGACATGCTCTGTGGCCCACCCTGCCTCTTCCACCA
CCGTGGACAAGAAGCTGGAACCCTCCGGCCCCATCTCCACCATCAACCCT
TGCCCTCCCTGCAAAGAATGCCACAAGTGCCCTGCCCCCAACCTGGAAGG
CGGCCCTTCCGTGTTCATCTTCCCACCCAACATCAAGGACGTGCTGATGA
TCTCCCTGACCCCCAAAGTGACCTGCGTGGTGGTGGACGTGTCCGAGGAC
GACCCTGACGTGCAGATCAGTTGGTTCGTGAACAACGTGGAAGTGCACAC
CGCCCAGACCCAGACACACAGAGAGGACTACAACAGCACCATCAGAGTGG
TGTCTACCCTGCCCATCCAGCACCAGGACTGGATGTCCGGCAAAGAATTC
AAGTGCAAAGTGAACAACAAGGACCTGCCCAGCCCCATCGAGCGGACCAT
CTCCAAGATCAAGGGCCTCGTGCGGGCTCCCCAGGTGTACATTCTGCCTC
CACCAGCCGAGCAGCTGTCCCGGAAGGATGTGTCTCTGACATGTCTGGTC
GTGGGCTTCAACCCCGGCGACATCTCCGTGGAATGGACCTCCAACGGCCA
CACCGAGGAAAACTACAAGGACACCGCCCCTGTGCTGGACTCCGACGGCT
CCTACTTCATCTACTCCAAGCTGAACATGAAGACCTCCAAGTGGGAAAAG
ACCGACTCCTTCTCCTGCAACGTGCGGCACGAGGGCCTGAAGAACTACTA
CCTGAAGAAAACCATCTCCCGGTCCCCCGGCTAG

The DNA sequence that corresponds to SEQ ID NO: 13 is shown below:

ATGGACCCCAAGGGCAGCCTGAGCTGGAGAATCCTGCTGTTCCTGAGCCT
GGCCTTCGAGCTGAGCTACGGCCAGGTGCAGCTGCTGCAGCCAGGTGCCG
AGCTCGTGAAACCTGGCGCCTCTGTGAAGCTGGCCTGCAAGGCTTCCGGC
TACCTGTTCACCACCTACTGGATGCACTGGCTGAAGCAGAGGCCAGGCCA
GGGCCTGGAATGGATCGGCGAGATCTCCCCCACCAACGGCAGAGCCTACT
ACAACGCCCGGTTCAAGTCCGAGGCCACCCTGACCGTGGACAAGTCCTCC
AACACCGCCTACATGCAGCTGTCCTCCCTGACCTCTGAGGCCTCCGCCGT
GTACTACTGCGCCAGAGCTTACGGCAACTACGAGTTCGCCTACTGGGGCC
AGGGCACCCTCGTGACAGTGTCTGTGGCTAGCACCAAGGGCCCCAGCGTG
TTCCCTCTGGCCCCCAGCAGCAAGAGCACCAGCGGCGGAACCGCCGCCCT
GGGCTGCCTGGTGAAGGACTACTTCCCCGAGCCCGTGACCGTGTCCTGGA
ACAGCGGCGCTCTGACCAGCGGAGTGCACACCTTCCCTGCCGTGCTGCAG
AGCAGCGGCCTGTACTCCCTGAGCAGCGTGGTGACCGTGCCCAGCAGCAG
CCTGGGCACCCAGACCTACATCTGCAACGTGAACCACAAGCCCTCCAACA
CCAAGGTGGACAAGAAGGTGGAGCCTAAGAGCTGCGACAAGACCCACACC
TGCCCTCCCTGCCCCGCCCCCGAGCTGCTGGGCGGACCCAGCGTGTTCCT
GTTCCCTCCCAAGCCCAAGGACACCCTGATGATCAGCCGCACCCCCGAGG
TGACCTGCGTGGTGGTGGACGTGAGCCACGAGGACCCCGAGGTGAAGTTC
AACTGGTACGTGGACGGCGTGGAGGTGCACAACGCCAAGACCAAGCCTCG
GGAGGAGCAGTACAACTCCACCTACCGCGTGGTGAGCGTGCTGACCGTGC
TGCACCAGGACTGGCTGAACGGCAAGGAGTACAAGTGCAAGGTGAGCAAC
AAGGCCCTGCCCGCTCCCATCGAGAAGACCATCAGCAAGGCCAAGGGCCA
GCCCCGGGAGCCTCAGGTGTACACCCTGCCCCCCAGCCGCGACGAGCTGA
CCAAGAACCAGGTGAGCCTGACCTGCCTGGTGAAGGGCTTCTACCCCTCC
GACATCGCCGTGGAGTGGGAGAGCAACGGCCAGCCTGAGAACAACTACAA
GACCACCCCTCCCGTGCTGGACAGCGACGGCAGCTTCTTCCTGTACAGCA
AGCTGACCGTGGACAAGTCCCGGTGGCAGCAGGGCAACGTGTTCAGCTGC
AGCGTGATGCACGAGGCCCTGCACAACCACTACACCCAGAAGAGCCTGAG
CCTGAGCCCCGGATAG

The DNA sequence that corresponds to SEQ ID NO: 14 is shown below:

ATGGAGACCGACACCCTGCTGCTCTGGGTGCTGCTGCTCTGGGTGCCCGG
CTCCACCGGAGACGTCGTGATGACCCAGACCCCTCTGTCCCTGCCTGTGT
CTCTGGGCGACCAGGCCTCCATCTCCTGCCGGTCTAGACAGTCCCTCGTG
AACTCCAACGGCAACACCTTCCTGCAGTGGTATCTGCAGAAGCCCGGCCA
GTCCCCCAAGCTGCTGATCTACAAGGTGTCCCTGCGGTTCTCCGGCGTGC
CCGACAGATTTTCCGGCTCTGGCTCTGGCACCGACTTCACCCTGAAGATC
TCCCGGGTGGAAGCCGAGGACCTGGGCCTGTACTTCTGCAGCCAGTCCAC
CCACGTGCCCCCTACATTTGGCGGAGGCACCAAGCTGGAAATCAAACGGG
CAGATGCTGCACCAACTGTATCCATCTTCCCACCATCCAGTGAGCAGTTA
ACATCTGGAGGTGCCTCAGTCGTGTGCTTCTTGAACAACTTCTACCCCAA
AGACATCAATGTCAAGTGGAAGATTGATGGCAGTGAACGACAAAATGGCG
TCCTGAACAGTTGGACTGATCAGGACAGCAAAGACAGCACCTACAGCATG
AGCAGCACCCTCACGTTGACCAAGGACGAGTATGAACGACATAACAGCTA
TACCTGTGAGGCCACTCACAAGACATCAACTTCACCCATTGTCAAGAGCT
TCAACAGGAATGAGTGTTGA

The DNA sequence that corresponds to SEQ ID NO: 15 is shown below:

ATGGAGACCGACACCCTGCTGCTCTGGGTGCTGCTGCTCTGGGTGCCCGG
CTCCACCGGAGACGTCGTGATGACCCAGACCCCTCTGTCCCTGCCTGTGT
CTCTGGGCGACCAGGCCTCCATCTCCTGCCGGTCTAGACAGTCCCTCGTG
AACTCCAACGGCAACACCTTCCTGCAGTGGTATCTGCAGAAGCCCGGCCA
GTCCCCCAAGCTGCTGATCTACAAGGTGTCCCTGCGGTTCTCCGGCGTGC
CCGACAGATTTTCCGGCTCTGGCTCTGGCACCGACTTCACCCTGAAGATC
TCCCGGGTGGAAGCCGAGGACCTGGGCCTGTACTTCTGCAGCCAGTCCAC
CCACGTGCCCCCTACATTTGGCGGAGGCACCAAGCTGGAAATCAAGCGGA
CCGTGGCCGCCCCCAGCGTGTTCATCTTCCCTCCCAGCGACGAGCAGCTG
AAGTCTGGCACCGCCAGCGTGGTGTGCCTGCTGAACAACTTCTACCCCCG
CGAGGCCAAGGTGCAGTGGAAGGTGGACAACGCCCTGCAGAGCGGCAACA
GCCAGGAGAGCGTGACCGAGCAGGACTCCAAGGACAGCACCTACAGCCTG
AGCAGCACCCTGACCCTGAGCAAGGCCGACTACGAGAAGCACAAGGTGTA
CGCCTGCGAGGTGACCCACCAGGGACTGTCTAGCCCCGTGACCAAGAGCT
TCAACCGGGGCGAGTGCTAA

The one-letter amino acid sequence that corresponds to SEQ ID NO: 16 is shown below:

MDPKGSLSWRILLFLSLAFELSYGQVQLLQPGAELVKPGASVKLACKASG
YLFTTYWMHWLKQRPGQGLEWIGEISPTNGRAYYNARFKSEATLTVDKSS
NTAYMQLSSLTSEASAVYYCARAYGNYEFAYWGQGTLVTVSVAKTTPPSV
YPLAPGCGDTTGSSVTLGCLVKGYFPESVTVTWNSGSLSSSVHTFPALLQ
SGLYTMSSSVTVPSSTWPSQTVTCSVAHPASSTTVDKKLEPSGPISTINP
CPPCKECHKCPAPNLEGGPSVFIFPPNIKDVLMISLTPKVTCVVVDVSED
DPDVQISWFVNNVEVHTAQTQTHREDYNSTIRVVSTLPIQHQDWMSGKEF
KCKVNNKDLPSPIERTISKIKGLVRAPQVYILPPPAEQLSRKDVSLTCLV
VGFNPGDISVEWTSNGHTEENYKDTAPVLDSDGSYFIYSKLNMKTSKWEK
TDSFSCNVRHEGLKNYYLKKTISRSPG*

The alanine residue at position 123 of the above amino acid sequence may optionally be replaced with a serine residue. The tyrosine residue at position 124 of the above amino acid sequence may optionally be replaced with a phenylalanine residue. Positions 25-142 of the above amino acid sequence correspond to SEQ ID NO: 20. SEQ ID NO: 20 may optionally include the substitutions at positions 123 and 124. SEQ ID NO: 20 may optionally contain one additional lysine residue after the terminal valine residue.

The one-letter amino acid sequence that corresponds to SEQ ID NO: 17 is shown below:

MDPKGSLSWRILLFLSLAFELSYGQVQLLQPGAELVKPGASVKLACKASG
YLFTTYWMHWLKQRPGQGLEWIGEISPTNGRAYYNARFKSEATLTVDKSS
NTAYMQLSSLTSEASAVYYCARAYGNYEFAYWGQGTLVTVSVASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPG*

The one-letter amino acid sequence that corresponds to SEQ ID NO: 18 is shown below:

METDTLLLWVLLLWVPGSTGDVVMTQTPLSLPVSLGDQASISCRSRQSLV
NSNGNTFLQWYLQKPGQSPKLLIYKVSLRFSGVPDRFSGSGSGTDFTLKI
SRVEAEDLGLYFCSQSTHVPPTFGGGTKLEIKRADAAPTVSIFPPSSEQL
TSGGASVVCFLNNFYPKDINVKWKIDGSERQNGVLNSWTDQDSKDSTYSM
SSTLTLTKDEYERHNSYTCEATHKTSTSPIVKSFNRNEC*

Positions 21-132 of the above amino acid sequence correspond to SEQ ID NO: 21.

The one-letter amino acid sequence that corresponds to SEQ ID NO: 19 is shown below:

METDTLLLWVLLLWVPGSTGDVVMTQTPLSLPVSLGDQASISCRSRQSLV
NSNGNTFLQWYLQKPGQSPKLLIYKVSLRFSGVPDRFSGSGSGTDFTLKI
SRVEAEDLGLYFCSQSTHVPPTFGGGTKLEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC*

The one-letter amino acid sequence that corresponds to SEQ ID NO: 22 is shown below:

```
                10 20 30 40
ASTKGPSVFP LAPCSRSTSE STAALGCLVK DYFPEPVTVS
                50 60 70 80
WNSGALTSGV HTFPAVLQSS GLYSLSSVVT VPSSNFGTQT
                90 100 110 120
YTCNVDHKPS NTKVDKTVER KCCVECPPCP APPVAGPSVF
              130 140 150 160
LFPPKPKDTL MISRTPEVTC VVVDVSHEDP EVQFNWYVDG
              170 180 190 200
VEVHNAKTKP REEQFNSTFR VVSVLTVVHQ DWLNGKEYKC
              210 220 230 240
KVSNKGLPAP IEKTISKTKG QPREPQVYTL PPSREEMTKN
              250 260 270 280
QVSLTCLVKG FYPSDISVEW ESNGQPENNY KTTPPMLDSD
              290 300 310 320
GSFFLYSKLT VDKSRWQQGN VFSCSVMHEA LHNHYTQKSL
SLSPGK
```

The one-letter amino acid sequence that corresponds to SEQ ID NO: 23 is SYTMGVS.

The one-letter amino acid sequence that corresponds to SEQ ID NO: 24 is

TISSGGGSTYYPDSVKG.

The one-letter amino acid sequence that corresponds to SEQ ID NO: 25 is QGGWLPPFAX, where X may be any naturally occurring amino acid.

The one-letter amino acid sequence that corresponds to SEQ ID NO: 26 is

RASKSVSTSSRGYSYMH.

The one-letter amino acid sequence that corresponds to SEQ ID NO: 27 is LVSNLES.

The one-letter amino acid sequence that corresponds to SEQ ID NO: 28 is

QHIRELTRS.

The one-letter amino acid sequence that corresponds to SEQ ID NO: 29 is

MDPKGSLSWRILLFLSLAFELSYGQVQLVQSGAEVKKPGASVKVSCKASG
YLFTTYWMHWVRQAPGQGLEWMGEISPTNGRAYYNQKFQGRVTMTVDKST
NTVYMELSSLRSEDTAVYYCARAYGNYFAYWGQGTLVTVSSASTKGPSVF
PLAPSSKSTSGGTAALGCLVKDYFPEPVTVSWNSGALTSGVHTFPAVLQS
SGLYSLSSWTVPSSSLGTQTYICNVNHKPSNTKVDKKVEPKSCDKTHTCP
PCPPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PAPIEKTISKAKGQPREPQVYTLPPSRDELKNQVSLTCLVKGFYPSDIAV
EWESNGQPENNYKTTPPVLDSDGSFFLYSKLTVDKSRWQQGNVFSCSVMH
EALHNHYTQKSLSLSPG.

The DNA sequence that corresponds to SEQ ID NO: 30 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACAGAAATTCCAGGGCAGAGTGACCATGACCGTGGACAAGTCCACCAA
CACCGTGTACATGGAACTGTCCTCCCTGCGGAGCGAGGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCTGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 31 is

MDPKGSLSWRILLFLSLAFELSYGQVQLVQSGAEVKKPGASVKVSCKASG
YLFTTYWMHVVVRQAPGQGLEWMGEISPTNGRAYYNAKFQGRVTMTVDKS
TNTAYMELSSLRSEDTAVYYCARAYGNYFAYWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPPELLGGPSVFLFPPKPKDTLMISRTPEVTCVVVDVSHEDPEVKFN
VVYVDGVEVHNAKTKPREEQYNSTYRVVSVLTVLHQDWLNGKEYKCKVSN
KALPAPIEKTISKAKGQPREPQVYTLPPSRDELKNQVSLTCLVKGFYPSD
IAVEWESNGQPENNYKTTPPVLDSDGSFFLYSKLTVDKSRWQQGNVFSCS
VMHEALHNHYTQKSLSLSPG.

The DNA sequence that corresponds to SEQ ID NO: 32 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACCAAAATTCCAGGGCAGAGTGACCATGACCGTGGACAAGTCCACCAA
CACCGCTTACATGGAACTGTCCTCCCTGCGGAGCGAGGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCTGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 33 is

MDPKGSLSWRILLFLSLAFELSYGQVQLVQSGAEVKKPGASVKVSCKASG
YLFTTYWMHVVVRQAPGQGLEWMGEISPTNGRAYYNAKFQGRVTMTVDKS
INTAYMELSRLRSDDTAVYYCARAYGNYFAYWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPPELLGGPSVFLFPPKPKDTLMISRTPEVTCVVVDVSHEDPEVKFN
VVYVDGVEVHNAKTKPREEQYNSTYRVVSVLTVLHQDWLNGKEYKCKVSN
KALPAPIEKTISKAKGQPREPQVYTLPPSRDELKNQVSLTCLVKGFYPSD
IAVEWESNGQPENNYKTTPPVLDSDGSFFLYSKLTVDKSRWQQGNVFSCS
VMHEALHNHYTQKSLSLSPG.

The DNA sequence that corresponds to SEQ ID NO: 34 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACCAAAATTCCAGGGCAGAGTGACCATGACCGTGGACAAGTCCATCAA
CACCGCTTACATGGAACTGTCCAGACTGCGGAGCGATGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCTGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 35 is

METDTLLLWVLLLWVPGSTGDVVMTQSPLSLPVTLGQPASISCRSSQSLV
NSNGNTFLQWYQQRPGQSPRLLIYKVSLRFSGVPDRFSGSGSGTDFTLKI
SRVEAEDVGVYYCSQSTHVPPTFGGGTVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEQ ID NO: 36 is

ATGGAGACCGACACCCTGCTGCTCTGGGTGCTGCTGCTCTGGGTGCCCGG
CTCCACCGGAGACGTCGTGATGACCCAGTCCCCTCTGTCCCTGCCTGTGA
CCCTGGGACAGCCTGCCTCCATCTCCTCAGATCCTCCCAGTCCCTCGTGA
ACTCCAACGGCAACACCTTCCTGCAGTGGTATCAGCAGCGGCCTGGCCAG
AGCCCCAGACTGCTGATCTACAAGGTGTCCCTGCGGTTCTCCGGCGTGCC
CGACGATTTTCCGGCTCTGGCTCTGGCACCGACTTCACCCTGAAGATCTC
CCGGGTGGAAGCCGAGGACGTGGGCGTGTACTACTGCTCCCAGAGCACCC
ACGTGCCCCCTACATTTGGCGGAGGCACCAAGTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

The one-letter amino acid sequence that corresponds to SEQ ID NO: 37 is

METDTLLLWVLLLWVPGSTGDVVMTQSPLSLPVTLGQPASISCRSRQSLV
NSNGNTFLQWYQQRPGQSPRLLIYKVSLRFSGVPDRFSGSGSGTDFTLKI
SRVEAEDVGVYYCSQSTHVPPTFGGGTVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEO ID NO: 38 is

ATGGAGACCGACACCCTGCTGCTCTGGGTGCTGCTGCTCTGGGTGCCCGG
CTCCACCGGAGACGTCGTGATGACCCAGTCCCCTCTGTCCCTGCCTGTGA
CCCTGGGACAGCCTGCCTCCATCTCCTCAGATCCAGGCAGTCCCTCGTGA
ACTCCAACGGCAACACCTTCCTGCAGTGGTATCAGCAGCGGCCTGGCCAG
AGCCCCAGACTGCTGATCTACAAGGTGTCCCTGCGGTTCTCCGGCGTGCC
CGACGATTTTCCGGCTCTGGCTCTGGCACCGACTTCACCCTGAAGATCTC
CCGGGTGGAAGCCGAGGACGTGGGCGTGTACTACTGCTCCCAGAGCACCC
ACGTGCCCCCTACATTTGGCGGAGGCACCAAGTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

The one-letter amino acid sequence that corresponds to SEQ ID NO: 39 is

METDTLLLWVLLLWVPGSTGDVVMTQSPLSSPVTLGQPASISCRSSQSLV
NSNGNTFLQWYHQRPGQPPRLLIYKVSLRFSGVPDRFSGSGAGKDFTLKI
SRVEAEDVGVYYCSQSTHVPPTFGQGTLEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEQ ID NO: 40 is

ATGGAGACCGACACCCTGCTGCTCTGGGTGCTGCTGCTCTGGGTGCCCGG
CTCCACCGGAGACGTCGTGATGACCCAGTCCCCTCTGTCCAGTCCTGTGA
CCCTGGGACAGCCTGCCTCCATCTCCTCAGATCCTCCCAGTCCCTCGTGA
ACTCCAACGGCAACACCTTCCTGCAGTGGTATCACCAGCGGCCTGGCCAG
CCTCCCAGACTGCTGATCTACAAGGTGTCCCTGCGGTTCTCCGGCGTGCC
CGACGATTTTCCGGCTCTGGCGCTGGCAAGGACTTCACCCTGAAGATCTC
CCGGGTGGAAGCCGAGGACGTGGGCGTGTACTACTGCTCCCAGAGCACCC
ACGTGCCCCCTACATTTGGCCAGGGCACCAACTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

### EXAMPLES

### Example 1: Affinity and Kinetics of Test Antibody

The affinity and kinetics of a test antibody were analyzed using Nα,Nα-bis(carboxymethyl)-L-lysine trifluoroacetate salt (Sigma-Aldrich, St. Louis, MO) as a model substrate for an AGE-modified protein. Label-free interaction analysis was carried out on a BIACORE™ T200 (GE Healthcare, Pittsburgh, PA), using a Series S sensor chip CM5 (GE Healthcare, Pittsburgh, PA), with Fc1 set as blank, and Fc2 immodilized with the test antibody (molecular weigh of 150,000 Da). The running buffer was a HBS-EP buffer (10 mM HEPES, 150 mM NaCl, 3 mM EDTA and 0.05% P-20, pH of 7.4), at a temperature of 25° C. Software was BIACORE™ T200 evaluation software, version 2.0. A double reference (Fc2-1 and only buffer injection), was used in the analysis, and the data was fitted to a Langmuir 1:1 binding model.

TABLE 1
Experimental set-up of affinity and kinetics analysis
Association and dissociation

| Flow path | Fc1 and Fc2 |
|---|---|
| Flow rate (μl/min.) | 30 |
| Association time (s) | 300 |
| Dissociation time (s) | 300 |
| Sample concentration (μM) | 20-5-1.25 (x2)-0.3125-0.078-0 |

A graph of the response versus time is illustrated in FIG. 1. The following values were determined from the analysis: ka (1/Ms)=1.857×10³; k_{d} (1/s)=6.781×10⁻³; Ko (M)=3.651×10⁻⁶; Rₘₐₓ (RU)=19.52; and Chi²=0.114. Because the Chi² value of the fitting is less than 10% of Rₘₐₓ, the fit is reliable.

### Example 2: Construction and Production of Murine Anti-AGE IgG2b Antibody and Chimeric Anti-AGE IgG1 Antibody

Murine and chimeric human anti-AGE antibodies were prepared. The DNA sequence of murine anti-AGE antibody IgG2b heavy chain is shown in SEQ ID NO: 12. The DNA sequence of chimeric human anti-AGE antibody IgG1 heavy chain is shown in SEQ ID NO: 13. The DNA sequence of murine anti-AGE antibody kappa light chain is shown in SEQ ID NO: 14. The DNA sequence of chimeric human anti-AGE antibody kappa light chain is shown in SEQ ID NO: 15. The gene sequences were synthesized and cloned into high expression mammalian vectors. The sequences were codon optimized. Completed constructs were sequence confirmed before proceeding to transfection.

HEK293 cells were seeded in a shake flask one day before transfection, and were grown using serum-free chemically defined media. The DNA expression constructs were transiently transfected into 0.03 liters of suspension HEK293 cells. After 20 hours, cells were sampled to obtain the viabilities and viable cell counts, and titers were measured (Octet QKe, ForteBio). Additional readings were taken throughout the transient transfection production runs. The cultures were harvested on day 5, and an additional sample for each was measured for cell density, viability and titer.

The conditioned media for murine and chimeric anti-AGE antibodies were harvested and clarified from the transient transfection production runs by centrifugation and filtration. The supernatants were run over a Protein A column and eluted with a low pH buffer. Filtration using a 0.2 μm membrane filter was performed before aliquoting. After purification and filtration, the protein concentrations were calculated from the OD280 and the extinction coefficient. A summary of yields and aliquots is shown in Table 2:

TABLE 2
Yields and Aliquots

| | Concentration | Volume | No. of | Total Yield |
|---|---|---|---|---|
| Protein | (mg/mL) | (mL) | vials | (mg) |
| Murine anti-AGE | 0.08 | 1.00 | 3 | 0.24 |
| Chimeric anti-AGE | 0.23 | 1.00 | 3 | 0.69 |

CE-SDS analysis was performed (LabChip GXII, Perkin Elmer) and the electropherograms were plotted.

### Example 3: Binding of Murine (Parental) and Chimeric Anti-AGE Antibodies

The binding of the murine (parental) and chimeric anti-AGE antibodies described in Example 2 was investigated by a direct binding ELISA. An anti-carboxymethyl lysine (CML) antibody (R&D Systems, MAB3247) was used as a control. CML was conjugated to KLH (CML-KLH) and both CML and CML-KLH were coated overnight onto an ELISA plate. HRP-goat anti-mouse Fc was used to detect the control and murine (parental) anti-AGE antibodies. HRP-goat anti-human Fc was used to detect the chimeric anti-AGE antibody.

The antigens were diluted to 1 μg/mL in 1× phosphate buffer at pH 6.5. A 96-well microtiter ELISA plate was coated with 100 μL/well of the diluted antigen and let sit at 4° C. overnight. The plate was blocked with 1×PBS, 2.5% BSA and allowed to sit for 1-2 hours the next morning at room temperature. The antibody samples were prepared in serial dilutions with 1×PBS, 1% BSA with the starting concentration of 50 μg/mL. Secondary antibodies were diluted 1:5,000. 100 μL of the antibody dilutions was applied to each well. The plate was incubated at room temperature for 0.5-1 hour on a microplate shaker. The plate was washed 3 times with 1×PBS. 100 μL/well diluted HRP-conjugated goat anti-human Fc secondary antibody was applied to the wells. The plate was incubated for 1 hour on a microplate shaker. The plate was then washed 3 times with 1×PBS. 100 μL HRP substrate TMB was added to each well to develop the plate. After 3-5 minutes elapsed, the reaction was terminated by adding 100 μL of 1N HCl. A second direct binding ELISA was performed with only CML coating. The absorbance at OD450 was read using a microplate reader.

The OD450 absorbance raw data for the CML and CML-KLH ELISA is shown in the plate map below. 48 of the 96 wells in the well plate were used. Blank wells in the plate map indicate unused wells.

Plate map of CML and CML-KLH ELISA:

| Conc. | | | | | | | |
|---|---|---|---|---|---|---|---|
| (μg/ | | | | | | | |
| mL) | 1 | 2 | 3 | 4 | 5 | 6 | 7 |
| 50 | 0.462 | 0.092 | 0.42 | | 1.199 | 0.142 | 1.852 |
| 16.67 | 0.312 | 0.067 | 0.185 | | 0.31 | 0.13 | 0.383 |
| 5.56 | 0.165 | 0.063 | 0.123 | | 0.19 | 0.115 | 0.425 |
| 1.85 | 0.092 | 0.063 | 0.088 | | 0.146 | 0.099 | 0.414 |
| 0.62 | 0.083 | 0.072 | 0.066 | | 0.108 | 0.085 | 0.248 |
| 0.21 | 0.075 | 0.066 | 0.09 | | 0.096 | 0.096 | 0.12 |
| 0.07 | 0.086 | 0.086 | 0.082 | | 0.098 | 0.096 | 0.098 |
| 0 | 0.09 | 0.085 | 0.12 | | 0.111 | 0.083 | 0.582 |
| | R&D | Parental | Chimeric | | R&D | Parental | Chimeric |
| | Positive | Anti- | Anti- | | Positive | Anti- | Anti- |
| | Control | AGE | AGE | | Control | AGE | AGE |

| | |
|---|---|
| CML-KLH Coat | CML Coat |

The OD450 absorbance raw data for the CML-only ELISA is shown in the plate map below. 24 of the 96 wells in the well plate were used. Blank wells in the plate map indicate unused wells.

| Conc. | | | | | | | |
|---|---|---|---|---|---|---|---|
| (μg/mL) | 1 | 2 | 3 | 4 | 5 | 6 | 7 |
| 50 | 1.913 | 0.165 | 0.992 | | | | |
| 16.66667 | 1.113 | 0.226 | 0.541 | | | | |
| 5.555556 | 0.549 | 0.166 | 0.356 | | | | |
| 1.851852 | 0.199 | 0.078 | 0.248 | | | | |
| 0.617284 | 0.128 | 0.103 | 0.159 | | | | |
| 0.205761 | 0.116 | 0.056 | 0.097 | | | | |
| 0.068587 | 0.073 | 0.055 | 0.071 | | | | |
| 0 | 0.053 | 0.057 | 0.06 | | | | |
| | R&D | Parental | Chimeric | | | | |
| | Positive | Anti- | Anti- | | | | |
| | Control | AGE | AGE | | | | |

The OD450 absorbance data was also plotted against antibody concentration.

The control and chimeric anti-AGE antibodies showed binding to both CML and CML-KLH. The murine (parental) anti-AGE antibody showed very weak to no binding to either CML or CML-KLH. Data from repeated ELISA confirms binding of the control and chimeric anti-AGE to CML. All buffer control showed negative signal.

### Example 4: Humanized Antibodies

Humanized antibodies were designed by creating multiple hybrid sequences that fuse select parts of the parental (mouse) antibody sequence with the human framework sequences. Acceptor frameworks were identified based on the overall sequence identity across the framework, matching interface position, similarly classed CDR canonical positions, and presence of N-glycosylation sites that would have to be removed. Three humanized light chains and three humanized heavy chains were designed based on two different heavy and light chain human acceptor frameworks. The amino acid sequences of the heavy chains are shown in SEQ ID NO: 29, 31 and 33, which are encoded by the DNA sequences shown in SEQ ID NO: 30, 32 and 34, respectively. The amino acid sequences of the light chains are shown in SEQ ID NO: 35, 37 and 39, which are encoded by the DNA sequences shown in SEQ ID NO: 36, 38 and 40, respectively. The humanized sequences were methodically analyzed by eye and computer modeling to isolate the sequences that would most likely retain antigen binding. The goal was to maximize the amount of human sequence in the final humanized antibodies while retaining the original antibody specificity. The light and heavy humanized chains could be combined to create nine variant fully humanized antibodies.

The three heavy chains and three light chains were analyzed to determine their humanness. Antibody humanness scores were calculated according to the method described in Gao, S. H., et al., “Monoclonal antibody humanness score and its applications”, BMC Biotechnology, 13:55 (Jul. 5, 2013). The humanness score represents how human-like an antibody variable region sequence looks. For heavy chains a score of 79 or above is indicative of looking human-like; for light chains a score of 86 or above is indicative of looking human-like. The humanness of the three heavy chains, three light chains, a parental (mouse) heavy chain and a parental (mouse) light chain are shown below in Table 3:

TABLE 3
Antibody humanness

| Antibody | Humanness (Framework + CDR) |
|---|---|
| Parental (mouse) heavy chain | 63.60 |
| Heavy chain 1 (SEQ ID NO: 29) | 82.20 |
| Heavy chain 2 (SEQ ID NO: 31) | 80.76 |
| Heavy chain 3 (SEQ ID NO: 33) | 81.10 |
| Parental (mouse) light chain | 77.87 |
| Light chain 1 (SEQ ID NO: 35) | 86.74 |
| Light chain 2 (SEQ ID NO: 37) | 86.04 |
| Light chain 3 (SEQ IN NO: 39) | 83.57 |

Full-length antibody genes were constructed by first synthesizing the variable region sequences. The sequences were optimized for expression in mammalian cells. These variable region sequences were then cloned into expression vectors that already contain human Fc domains; for the heavy chain, the IgG1 was used.

Small scale production of humanized antibodies was carried out by transfecting plasmids for the heavy and light chains into suspension HEK293 cells using chemically defined media in the absence of serum. Whole antibodies in the conditioned media were purified using MabSelect SuRe Protein A medium (GE Healthcare).

Nine humanized antibodies were produced from each combination of the three heavy chains having the amino acid sequences shown in SEQ ID NO: 29, 31 and 33 and three light chains having the amino acid sequences shown in SEQ ID NO: 35, 37 and 39. A comparative chimeric parental antibody was also prepared. The antibodies and their respective titers are shown below in Table 4:

TABLE 4
The antibodies and their respective titers

| Antibody | Titer (mg/L) |
|---|---|
| Chimeric parental | 23.00 |
| SEQ ID NO: 29 + SEQ ID NO: 35 | 24.67 |
| SEQ ID NO: 29 + SEQ ID NO: 37 | 41.67 |
| SEQ ID NO: 29 + SEQ ID NO: 39 | 29.67 |
| SEQ ID NO: 31 + SEQ ID NO: 35 | 26.00 |
| SEQ ID NO: 31 + SEQ ID NO: 37 | 27.33 |
| SEQ ID NO: 31 + SEQ ID NO: 39 | 35.33 |
| SEQ ID NO: 33 + SEQ ID NO: 35 | 44.00 |
| SEQ ID NO: 33 + SEQ ID NO: 37 | 30.33 |
| SEQ ID NO: 33 + SEQ ID NO: 39 | 37.33 |

The binding of the humanized antibodies may be evaluated, for example, by dose-dependent binding ELISA or cell-based binding assay.

### Example 5: Immunohistochemical Study

Tissue samples were obtained from patients with Alzheimer's disease and Parkinson's disease. Two Alzheimer's disease samples were taken from the hippocampus. One Parkinson's disease sample was taken from the substantia nigra, and a second Parkinson's disease sample was taken from the ventral tegmental area. All cells were stained for carboxymethyllysine (CML) using anti-AGE antibodies as described above. The Alzheimer's disease cells were stained for phosphorylated tau (phospho tau) or separately amyloid precursor protein. The Parkinson's disease cells were stained for alpha synuclein. Nuclear staining of the cells was identified using DAPI counter stain. (Experiments were carried out and images were prepared by Dr. Diego Mastroeni of Arizona State University.)

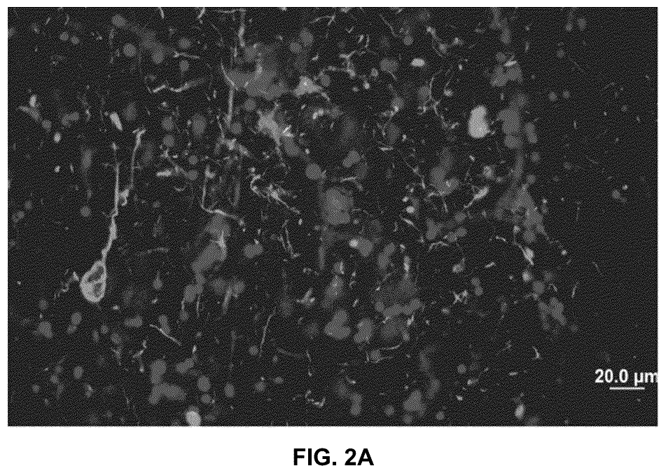

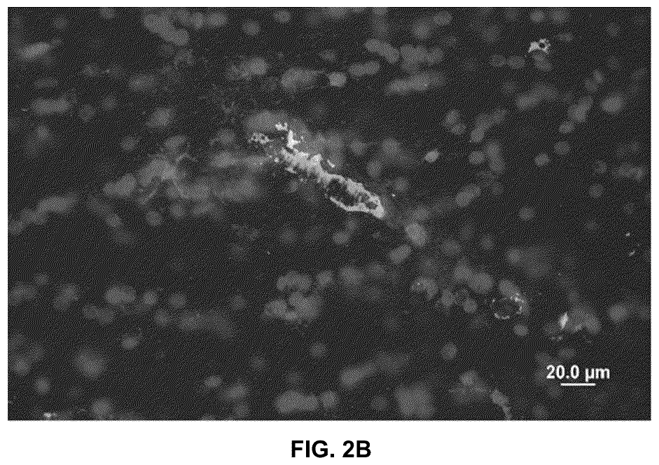

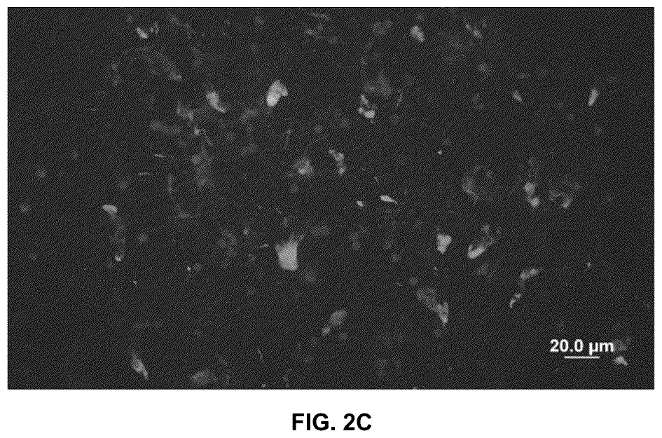

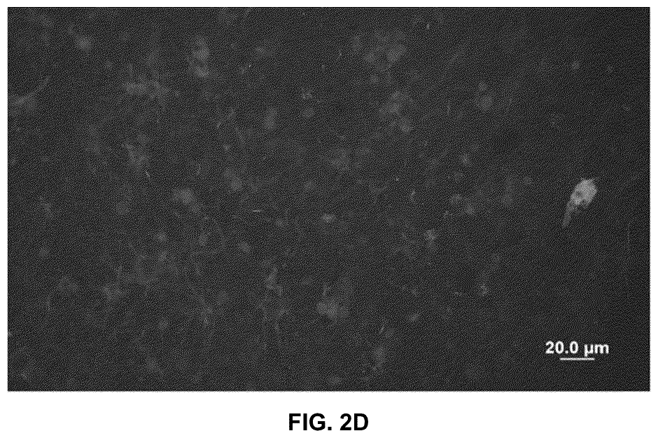

FIG. 2A is a photograph of cells of the Alzheimer's disease sample showing carboxymethyllysine stained gray and phosphorylated tau stained light gray (CML (red) Phospho TAU (green) Hippocampus AD).

FIG. 2B is a photograph of cells of the Alzheimer's disease sample showing carboxymethyllysine stained gray and amyloid precursor protein stained light gray (CML (red) Amyloid Precursor Protein (green) Hippocampus AD).

FIG. 2C is a photograph of cells of the Parkinson's disease sample from the substantia nigra showing carboxymethyllysine stained gray and alpha synuclein stained light gray (CML (red) Alpha Synuclein (green) Substantia Nigra PD).

FIG. 2D is a photograph of cells of the Parkinson's disease sample from the ventral tegmental area showing carboxymethyllysine stained gray and alpha synuclein stained light gray (CML (red) Alpha Synuclein (green) Ventral Tegmental Area PD).

CML, a well-known AGE, did not co-localize with established pathologies in Alzheimer's disease and Parkinson's disease. Instead, the CML presented on glial cells. It was suspected that the CML immunoreactivity in the Alzheimer's disease samples was with microglia, and the CML immunoreactivity in the Parkinson's disease samples was with astrocytes. The results demonstrate the presence of senescent glial cells in Alzheimer's disease and Parkinson's disease. Removal of senescent glial cells using an anti-AGE antibody would be expected to result in regeneration of the glial cells by neural stem/progenitor cells. (See, for example, Leonard, B. W. et al., “Subventricular zone neural progenitors from rapid brain autopsies of elderly subjects with and without neurodegenerative disease”, The Journal of Comparative Neurology, Vol. 515, pp. 269-294 (2009)).

### REFERENCES

1. International Application Pub. No. WO 2009/143411 to Gruber (26 Nov. 2009).
2. U.S. Pat. No. 5,702,704 to Bucala (issued Dec. 30, 1997).
3. U.S. Pat. No. 6,380,165 to Al-Abed et al. (issued Apr. 30, 2002).
4. U.S. Pat. No. 6,387,373 to Wright et al. (issued May 14, 2002).
5. U.S. Pat. No. 4,217,344 to Vanlerberghe et al. (issued Aug. 12, 1980).
6. U.S. Pat. No. 4,917,951 to Wallach (issued Apr. 17, 1990).
7. U.S. Pat. No. 4,911,928 to Wallach (issued Mar. 27, 1990).
8. U.S. Patent Application Publication Pub. No. US 2010/226932 to Smith et al. (Sep. 9, 2010).
9. Ando K, et al., “Membrane Proteins of Human Erythrocytes Are Modified by Advanced Glycation End Products During Aging in the Circulation,” Biochemical and Biophysical Research Communications, Vol. 258, 123-27 (1999).
10. Lindsey J B, et al., “Receptor For Advanced Glycation End-Products (RAGE) and soluble RAGE (sRAGE): Cardiovascular Implications,” Diabetes Vascular Disease Research, Vol. 6(1), 7-14, (2009).
11. Bierhaus A, “AGEs and their interaction with AGE-receptors in vascular disease and diabetes mellitus. I. The AGE concept,” Cardiovasc Res, Vol. 37(3), 586-600 (1998).
12. Meuter A., et al. “Markers of cellular senescence are elevated in murine blastocysts cultured in vitro: molecular consequences of culture in atmospheric oxygen” J Assist Reprod Genet. 2014 Aug. 10. [Epub ahead of print].
13. Baker, D. J. et al., “Clearance of p16Ink4a-positive senescent cells delays ageing-associated disorders”, Nature, vol. 479, pp. 232-236, (2011).
14. Jana Hadrabove, et al. “Chicken immunoglobulins for prophylaxis: Effect of inhaled antibodies on inflammatory parameters in rat airways” Journal of Applied Biomedicine (in press; Available online 5 May 2014).
15. Vlassara, H. et al., “High-affinity-receptor-mediated Uptake and Degradation of Glucose-modified Proteins: A Potential Mechanism for the Removal of Senescent Macromolecules”, Proc. Natl. Acad. Sci. USA, Vol. 82, 5588, 5591 (1985).
16. Roll, P. et al., “Anti-CD20 Therapy in Patients with Rheumatoid Arthritis”, Arthritis & Rheumatism, Vol. 58, No. 6, 1566-1575 (2008).
17. Kajstura, J. et al., “Myocite Turnover in the Aging Human Heart”, Circ. Res., Vol. 107(11), 1374-86, (2010).
18. de Groot, K. et al., “Vascular Endothelial Damage and Repair in Antineutrophil Cytoplasmic Antibody-Associated Vasculitis”, Arthritis and Rheumatism, Vol. 56(11), 3847, 3847 (2007).
19. Manesso, E. et al., “Dynamics of β-Cell Turnover: Evidence for β-Cell Turnover and Regeneration from Sources of β-Cells other than β-cell Replication in the HIP Rat”, Am. J. Physiol. Endocrinol. Metab., Vol. 297, E323, E324 (2009).
20. Kirstein, M. et al., “Receptor-specific Induction of Insulin-like Growth Factor I in Human Monocytes by Advanced Glycosylation End Product-modified Proteins”, J. Clin. Invest., Vol. 90, 439, 439-440 (1992).
21. Murphy, J. F., “Trends in cancer immunotherapy”, Clinical Medical Insights: Oncology, Vol. 14(4), 67-80 (2010).
22. Virella, G. et al., “Autoimmune Response to Advanced Glycosylation End-Products of Human LDL”, Journal of Lipid Research, Vol. 44, 487-493 (2003).
23. Ameli, S. et al., “Effect of Immunization With Homologous LDL and Oxidized LDL on Early Atherosclerosis in Hypercholesterolemic Rabbits”, Arteriosclerosis, Thrombosis, and Vascular Biology, Vol. 16, 1074 (1996).
24. “Sarcopenia”, available online at en.wikipedia.org/wiki/Sarcopenia (Nov. 14, 2014).
25. “What is sarcopenia?”, available online at www.iofbonehealth.org/what-sarcopenia (2014).
26. Blahd, W., “Sarcopenia with aging”, available online at www.webmd.com/healthy-aging/sarcopenia-with-aging (Aug. 3, 2014).
27. “Keyhole limpet hemocyanin”, available online at en.wikipedia.org/wiki/Keyhole_limpet_hemocyanin (Apr. 18, 2014).
28. “CML-BSA Product Data Sheet”, available online at www.cellbiolabs.com/sites/default/files/STA-314-cml-bsa.pdf (2010).
29. “CML (N-epsilon-(Carboxymethyl)Lysine) Assays and Reagents”, available online at www.cellbiolabs.com/cml-assays (Accessed on Dec. 15, 2014).
30. Cruz-Jentoft, A. J. et al., “Sarcopenia: European consensus on definition and diagnosis”, Age and Ageing, Vol. 39, pp. 412-423 (Apr. 13, 2010).
31. Rolland, Y. et al., “Sarcopenia: its assessment, etiology, pathogenesis, consequences and future perspectives”, J. Nutr. Health Aging, Vol. 12(7), pp. 433-450 (2008).
32. Mera, K. et al., “An autoantibody against N^{ε}-(carboxyethyl)lysine (CEL): Possible involvement in the removal of CEL-modified proteins by macrophages”, Biochemical and Biophysical Research Communications, Vol. 407, pp. 420-425 (Mar. 12, 2011).
33. Reddy, S. et al., “N^{ε}-(carboxymethyl)lysine is a dominant advanced glycation end product (AGE) antigen in tissue proteins”, Biochemistry, Vol. 34, pp. 10872-10878 (Aug. 1, 1995).
34. Naylor, R. M. et al., “Senescent cells: a novel therapeutic target for aging and age-related diseases”, Clinical Pharmacology & Therapeutics, Vol. 93(1), pp. 105-116 (Dec. 5, 2012).
35. Katcher, H. L., “Studies that shed new light on aging”, Biochemistry (Moscow), Vol. 78(9), pp. 1061-1070 (2013).
36. Ahmed, E. K. et al., “Protein Modification and Replicative Senescence of WI-38 Human Embryonic Fibroblasts”, Aging Cells, Vol. 9, 252, 260 (2010).
37. Vlassara, H. et al., “Advanced Glycosylation Endproducts on Erythrocyte Cell Surface Induce Receptor-Mediated Phagocytosis by Macrophages”, J. Exp. Med., Vol. 166, 539, 545 (1987).
38. Fielding, R. A., et al., “Sarcopenia: an undiagnosed condition in older adults. Current consensus definition: prevalence, etiology, and consequences”, Journal of the American Medical Directors Association, Vol. 12(4), pp. 249-256 (May 2011).
39. Maass, D. R. et al., “Alpaca (Lama pacos) as a convenient source of recombinant camelid heavy chain antibodies (VHHs)”, Journal of Immunological Methods, Vol. 324, No. 1-2, pp. 13-25 (Jul. 31, 2007).
40. Strietzel, C. J. et al., “In vitro functional characterization of feline IgGs”, Veterinary Immunology and Immunopathology, Vol. 158, pp. 214-223 (2014).
41. Patel, M. et al., “Sequence of the dog immunoglobulin alpha and epsilon constant region genes”, Immunogenetics, Vol. 41, pp. 282-286 (1995).
42. Wagner, B. et al., “The complete map of the Ig heavy chain constant gene region reveals evidence for seven IgG isotypes and for IgD in the horse”, The Journal of Immunology, Vol. 173, pp. 3230-3242 (2004).
43. Hamers-Casterman, C. et al., “Naturally occurring antibodies devoid of light chains”, Nature, Vol. 363, pp. 446-448 (Jun. 3, 1993).
44. De Genst, E. et al., “Antibody repertoire development in camelids”, Developmental & Comparative Immunology, Vol. 30, pp. 187-198 (available online Jul. 11, 2005).
45. Griffin, L. M. et al., “Analysis of heavy and light chain sequences of conventional camelid antibodies from Camelus dromedarius and Camelus bactrianus species”, Journal of Immunological Methods, Vol. 405, pp. 35-46 (available online Jan. 18, 2014).
46. Nguyen, V. K. et al., “Camel heavy-chain antibodies: diverse germline V_{H}H and specific mechanisms enlarge the antigen-binding repertoire”, The European Molecular Biology Organization Journal, Vol. 19, No, 5, pp. 921-930 (2000).
47. Muyldermans, S. et al., “Sequence and structure of V_{H} domain from naturally occurring camel heavy chain immunoglobulins lacking light chains”, Protein Engineering, Vol. 7, No. 9, pp. 1129-1135 (1994).
48. Wesolowski, J. et al., “Single domain antibodies: promising experimental and therapeutic tools in infection and immunity”, Medical Microbiology and Immunology, Vol. 198, pp. 157-174 (Jun. 16, 2009).
49. Yan, S. F. et al., “Soluble RAGE: therapy & biomarker in unraveling the RAGE axis in chronic disease and aging”, Biochemical Pharmacology, Vol. 79, No. 10, pp. 1379-1386 (May 15, 2010).
50. Chen, K. S. et al., “Monoclonal antibody therapy for malignant glioma”, Glioma: Immunotherapeutic Approaches, pp. 121-141 (2012).
51. Gao, S. H., et al., “Monoclonal antibody humanness score and its applications”, BMC Biotechnology, 13:55 (Jul. 5, 2013).
52. Feige, M. J. et al., “The structural analysis of shark IgNAR antibodies reveals evolutionary principles of immunoglobulins”, Proceedings of the National Academy of Sciences of the United States of America, Vol. 111, No. 22, pp. 8155-8160 (Jun. 3, 2014).
53. Romagosa, C. et al., p16^{Ink4a} overexpression in cancer: a tumor suppressor gene associated with senescence and high-grade tumors, Oncogene, Vol. 30, 2087-2097 (2011).
54. Di, G-h. et al. IL-6 Secreted from Senescent Mesenchymal Stem Cells Promotes Proliferation and migration of Breast Cancer Cells, PLOS One, Vol. 9, 11, e113572 (2014)
55. Liu, D. et al. Senescent Human Fibroblasts Increase the Early Growth of Xenograft Tumors via Matrix Metalloproteinase Secretion, Cancer Res, Vol. 67, 3117-3126 (2007)
56. Nelson, G., A senescent cell bystander effect: senescence-induced senescence, Aging Cell, Vo. 11, 345-349 (2012)
57. Rayess, H. et al., Cellular senescence and tumor suppressor gene p16, Int J Cancer, Vol. 130, 1715-1725 (2012)
58. Fu, M.-X., et al., The Advanced Glycation End Product, N^{E}-(Carboxymehtyl)lysine, Is a Product of both Lipid Peroxidation and Glycoxidation Reactions, J. Biol. Chem., Vol. 271, 9982-9986 (1996)
59. Matus et al., Invasive Cell Fate Requires G1 Cell-Cycle arrest and Histone Deacetylase-Mediated Changes in Gene Expression, Developmental Cell, Vol. 35, 162-174 (2015)
60. Blasko, C. et al., “Glial cells: astrocytes and oligodendrocytes during normal brain aging”, Encyclopedia of Neuroscience, Academic Press, pp. 743-747 (2009)
61. Danysz, W. et al., “Alzheimer's disease, β-amyloid, glutamate, NMDA receptors and memantine—searching for the connections”, British Journal of Pharmacology, Vol. 167, pp. 324-352 (2012)
62. Garcia-Matas, S. et al., “Dysfunction of astrocytes in senescence-accelerated mice SAMP8 reduces their neuroprotective capacity”, Aging Cell, Vol. 7, pp. 630-640 (2008)
63. Nie, H. et al., “Impaired glial glutamate uptake induces extrasynaptic glutamate spillover in the spinal sensory synapses of neuropathic rats”, Journal of Neurophysiology, Vol. 103, pp. 2570-2580 (2010)
64. Leonard, B. W. et al., “Subventricular zone neural progenitors from rapid brain autopsies of elderly subjects with and without neurodegenerative disease”, The Journal of Comparative Neurology, Vol. 515, pp. 269-294 (2009)

What is claimed is:

1. A method of treating a neurodegenerative disorder or MD, comprising administering to a subject a composition comprising an AGE antibody.

2. A method of killing senescent glial cells, senescent myoblasts and/or senescent myosatellite cells, comprising administering to a subject a composition comprising an AGE antibody.

3. (canceled)

4. The method of claim 1, further comprising:
testing the subject for effectiveness of the first administration at treating the neurodegenerative disorder or MD; followed by
a second administering of the AGE antibody.

5. The method of claim 1, wherein the AGE antibody binds to and kills or induces apoptosis in senescent glial cells, senescent myoblasts and/or senescent myosatellite cells.

6. A composition for treating a neurodegenerative disorder, comprising:
(i) an AGE antibody,
(ii) serum, immune system cells, or both.

7. The method of claim 1, wherein the composition further comprises a pharmaceutically acceptable carrier.

8. The method of claim 1, wherein the subject is selected from the group consisting of humans, mice, rats, goats, sheep, cows, horses, dogs and cats.

9. (canceled)

10. The method of claim 1, wherein the AGE antibody is non-immunogenic to a species selected from the group consisting of humans, cats, dogs, horses, camels, alpaca, cattle, sheep, and goats

11. The method of claim 1, wherein the AGE antibody binds an AGE antigen comprising at least one protein or peptide that exhibits AGE modifications selected from the group consisting of FFI, pyrraline, AFGP, ALI, carboxymethyllysine, carboxyethyllysine and pentosidine.

12. The method of claim 1, wherein the AGE antibody binds a carboxymethyllysine-modified protein.

13-20. (canceled)

21. The method of claim 1, wherein the composition further comprises:
(i) a pharmaceutically acceptable carrier,
(ii) serum, and
(iii) natural killer cells.

22. The method of claim 1, wherein the subject has a neurodegenerative disorder selected from the group consisting of AD, PD, Lewy body dementia, MS, prion diseases and ALS.

23. (canceled)

24. The method of claim 1, wherein the administering comprises administering the composition to the central nervous system of the subject.

25. (canceled)

26. The method of claim 1, wherein the subject has ALS or MD and the administering comprises administering the composition to muscles of the subject.

27-30. (canceled)

31. The composition of claim 6, wherein the composition comprises the serum and the immune system cells.

32-34. (canceled)

35. The method of claim 1, wherein the AGE antibody is a single domain antibody conjugated to an agent that causes the destruction of cells.

36. The method of claim 1, wherein the neurodegenerative disorder comprises Alzheimer's disease or Parkinson's disease.

37. The method of claim 5, wherein the neurodegenerative disorder comprises Alzheimer's disease or Parkinson's disease.

38. The method of claim 2, wherein the AGE antibody binds a carboxymethyllysine-modified protein.

39. The composition of claim 6, wherein the AGE antibody binds a carboxymethyllysine-modified protein.


SEQUENCE LISTING (40 sequences)

SEQ ID NO: 1 - protein, 463 aa, synthetic construct
  REGION 1..463: note = Modified Homo sapiens immunoglobulin G1 heavy chain
MNLLLILTFVAAAVAQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSV

SEQ ID NO: 3 - protein, 234 aa, synthetic construct
  REGION 1..234: note = Modified Homo sapiens immunoglobulin G1 kappa light chain
MNLLLILTFVAAAVADVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Mus musculus
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKR

SEQ ID NO: 5 - protein, 327 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 6 - protein, 415 aa, Equus caballus
SLEDTAVIPLFSECKAPKEDDVVSLACLVKGYFPEPVQVTWEPEMQNQKPWTFPAMKKGQEYIHVFSLTTWWKPGSHSCTVHHKASSFRKKMTFQEPASWAPQRTSALPVTSKEPTPAPTTLRKSEPSTRHTQPETQKPRIPVDTPLKECQSHTHPPSIYLLHPPLQGLWLKGEATFTCLVVGDDLKDAHLSWELSERSNGMFVESGPLEKHTNGSQSRSSRLALPRSSWAMGTSVTCKLSYPNLLSSMEVVGLKEHAASAPRSLTVHALTTPGLNASPGATSWLQCKVSGFSPPEIVLTWLEGQREVDPSWFATARPTAQPGNTTFQTWSILLVPTIPGPPTATYTCVVGHEASRQLLNTSWSLDTGGLAMTPESKDENSDDYADLDDAGSLWLTFMALFLITLLYSGFVTFIK

SEQ ID NO: 7 - protein, 334 aa, Canis sp.
  source 1..334: note = Canis familiaris (subspecies)
SKTSPSVFPLSLCHQESEGYVVIGCLVQGFFPPEPVNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSNASLTCTLSGLKDPKGATFTWNPSKGKEPIQKNPERDSCGCYSVSSVLPGCADPWNHGDTFSCTATHPESKSPITVSITKTTEHIPPQVHLLPPPSEELALNELVTLTCLVRGFKPKDVLVRWLQGTQELPQEKYLTWEPLKEPDQTNMFAVTSMLRVTAEDWKQGEKFSCMVGHEALPMSFTQKTIDRLAGKPTHVNVSVV

SEQ ID NO: 8 - protein, 426 aa, Canis sp.
  source 1..426: note = Canis familiaris (subspecies)
TSQDLSVFPLASCCKDNIASTSVTLGCLVTGYLPMSTTVTWDTGSLNKNVTTFPTTFHETYGLHSIVSQVTASGKWAKQRFTCSVAHAESTAINKTFSACALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 9 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 10 - protein, 96 aa, Camelus dromedarius
EVQLVESGGGLVQPGGSLRLSCAASGFTFSSYDMSWVRQAPGREREGVAAINSGGGSTYYADSVKGRFTISQDNAKNTVYLQMNSLKPEDTAMYYC

SEQ ID NO: 11 - protein, 96 aa, Camelus dromedarius
EVQLVESGGGLVQPGGSLRLSCAASGFTFSSYWMYWVRQAPGKGLEWVSTINSGGGSTYYADSVKGRFTISRDNAKNMLYLQMNSLKPEDTAMYYC

SEQ ID NO: 12 - DNA, 1434 bp, synthetic construct
  misc_feature 1..1434: note = Murine anti-AGE IgG2b heavy chain
  source 1..1434: mol_type = other DNA
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctaagaccacccctccctccgtgtaccctctggctcctggctgtggcgacaccaccggatcctctgtgaccctgggctgcctcgtgaagggctacttccctgagtccgtgaccgtgacctggaactccggctccctgtcctcctccgtgcacacctttccagccctgctgcagtccggcctgtacaccatgtcctccagcgtgacagtgccctcctccacctggccttcccagaccgtgacatgctctgtggcccaccctgcctcttccaccaccgtggacaagaagctggaaccctccggccccatctccaccatcaacccttgccctccctgcaaagaatgccacaagtgccctgcccccaacctggaaggcggcccttccgtgttcatcttcccacccaacatcaaggacgtgctgatgatctccctgacccccaaagtgacctgcgtggtggtggacgtgtccgaggacgaccctgacgtgcagatcagttggttcgtgaacaacgtggaagtgcacaccgcccagacccagacacacagagaggactacaacagcaccatcagagtggtgtctaccctgcccatccagcaccaggactggatgtccggcaaagaattcaagtgcaaagtgaacaacaaggacctgcccagccccatcgagcggaccatctccaagatcaagggcctcgtgcgggctccccaggtgtacattctgcctccaccagccgagcagctgtcccggaaggatgtgtctctgacatgtctggtcgtgggcttcaaccccggcgacatctccgtggaatggacctccaacggccacaccgaggaaaactacaaggacaccgcccctgtgctggactccgacggctcctacttcatctactccaagctgaacatgaagacctccaagtgggaaaagaccgactccttctcctgcaacgtgcggcacgagggcctgaagaactactacctgaagaaaaccatctcccggtcccccggctag

SEQ ID NO: 13 - DNA, 1416 bp, synthetic construct
  misc_feature 1..1416: note = Chimeric anti-AGE human IgG1 antibody heavy chain
  source 1..1416: mol_type = other DNA
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 14 - DNA, 720 bp, synthetic construct
  misc_feature 1..720: note = Murine anti-AGE Kappa light chain
  source 1..720: mol_type = other DNA
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 15 - DNA, 720 bp, synthetic construct
  misc_feature 1..720: note = Chimeric anti-AGE human kappa light chain
  source 1..720: mol_type = other DNA
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 16 - protein, 477 aa, synthetic construct
  REGION 1..477: note = Murine anti-AGE IgG2b heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 17 - protein, 471 aa, synthetic construct
  REGION 1..471: note = Chimeric anti-AGE human IgG1 heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 239 aa, synthetic construct
  REGION 1..239: note = Murine anti-AGE kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 239 aa, synthetic construct
  REGION 1..239: note = Chimeric anti-AGE human kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, synthetic construct; identical to SEQ ID NO: 2
  REGION 1..118: note = Murine anti-AGE IgG2b heavy chain (variable region)

SEQ ID NO: 21 - protein, 112 aa, synthetic construct
  REGION 1..112: note = Murine anti-AGE kappa light chain (variable region)
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIK

SEQ ID NO: 22 - protein, 326 aa, synthetic construct
  REGION 1..326: note = Human constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 23 - protein, 7 aa, synthetic construct
  REGION 1..7: note = CDR1H (heavy chain)
SYTMGVS

SEQ ID NO: 24 - protein, 17 aa, synthetic construct
  REGION 1..17: note = CDR2H (heavy chain)
TISSGGGSTYYPDSVKG

SEQ ID NO: 25 - protein, 10 aa, synthetic construct
  REGION 1..10: note = CDR3H (heavy chain)
  VAR_SEQ 10: note = misc_feature - Xaa can be any naturally occurring amino acid
QGGWLPPFAX

SEQ ID NO: 26 - protein, 17 aa, synthetic construct
  REGION 1..17: note = CDR1L (light chain)
RASKSVSTSSRGYSYMH

SEQ ID NO: 27 - protein, 7 aa, synthetic construct
  REGION 1..7: note = CDR2L (light chain)
LVSNLES

SEQ ID NO: 28 - protein, 9 aa, synthetic construct
  REGION 1..9: note = CDR3L (light chain)
QHIRELTRS

SEQ ID NO: 29 - protein, 468 aa, synthetic construct
  REGION 1..468: note = Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNQKFQGRVTMTVDKSTNTVYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 1408 bp, synthetic construct
  misc_feature 1..1408: note = Humanized heavy chain
  source 1..1408: mol_type = other DNA
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaacagaaattccagggcagagtgaccatgaccgtggacaagtccaccaacaccgtgtacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 31 - protein, 468 aa, synthetic construct
  REGION 1..468: note = Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSTNTAYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 1408 bp, synthetic construct
  misc_feature 1..1408: note = Humanized heavy chain
  source 1..1408: mol_type = other DNA
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccaccaacaccgcttacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 33 - protein, 468 aa, synthetic construct
  REGION 1..468: note = Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSINTAYMELSRLRSDDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 1408 bp, synthetic construct
  misc_feature 1..1408: note = Humanized heavy chain
  source 1..1408: mol_type = other DNA
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccatcaacaccgcttacatggaactgtccagactgcggagcgatgacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 35 - protein, 238 aa, synthetic construct
  REGION 1..238: note = Humanized light chain
METDTLLLWVLLLWVPGSTGDVVMTQSPLSLPVTLGQPASISCRSSQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 715 bp, synthetic construct
  misc_feature 1..715: note = Humanized light chain
  source 1..715: mol_type = other DNA
atggagaccgacaccctgctgctctgggtgctgctgctctgggtgcccggctccaccggagacgtcgtgatgacccagtcccctctgtccctgcctgtgaccctgggacagcctgcctccatctcctcagatcctcccagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 37 - protein, 238 aa, synthetic construct
  REGION 1..238: note = Humanized light chain
METDTLLLWVLLLWVPGSTGDVVMTQSPLSLPVTLGQPASISCRSRQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 715 bp, synthetic construct
  misc_feature 1..715: note = Humanized light chain
  source 1..715: mol_type = other DNA
atggagaccgacaccctgctgctctgggtgctgctgctctgggtgcccggctccaccggagacgtcgtgatgacccagtcccctctgtccctgcctgtgaccctgggacagcctgcctccatctcctcagatccaggcagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 39 - protein, 238 aa, synthetic construct
  REGION 1..238: note = Humanized light chain
METDTLLLWVLLLWVPGSTGDVVMTQSPLSSPVTLGQPASISCRSSQSLVNSNGNTFLQWYHQRPGQPPRLLIYKVSLRFSGVPDRFSGSGAGKDFTLKISRVEAEDVGVYYCSQSTHVPPTFGQGTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 715 bp, synthetic construct
  misc_feature 1..715: note = Humanized light chain
  source 1..715: mol_type = other DNA
atggagaccgacaccctgctgctctgggtgctgctgctctgggtgcccggctccaccggagacgtcgtgatgacccagtcccctctgtccagtcctgtgaccctgggacagcctgcctccatctcctcagatcctcccagtccctcgtgaactccaacggcaacaccttcctgcagtggtatcaccagcggcctggccagcctcccagactgctgatctacaaggtgtccctgcggttctccggcgtgcccgacgattttccggctctggcgctggcaaggacttcaccctgaagatctcccgggtggaagccgaggacgtgggcgtgtactactgctcccagagcacccacgtgccccctacatttggccagggcaccaactggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa